(19) United States
(12) Patent Application Publication
(10) Pub. No.: US 2023/0124836 A1
(43) Pub. Date: Apr. 20, 2023

(54) SYSTEM USING A METHOD FOR SEARCHING AND IDENTIFYING A GENETIC CONDITION PRODROMAL OF THE ONSET OF SOLID TUMORS

(71) Applicant: BIOSCIENCE SERVICES S.R.L., Falciano (SM)

(72) Inventor: Giuseppe Mucci, Falciano (SM)

(21) Appl. No.: 17/822,484

(22) Filed: Aug. 26, 2022

Related U.S. Application Data
(62) Division of application No. 16/607,993, filed on Oct. 24, 2019.

(30) Foreign Application Priority Data
Apr. 26, 2017 (IT) 102017000045353

(51) Int. Cl. C12Q 1/6886; G16B 20/20; G16B 20/50

(52) U.S. Cl. CPC C12Q 1/6886; G16B 20/20; G16B 20/50; C12Q 2600/156

(57) ABSTRACT

A system is shown that performs a method that searches for and identifies a genetic condition prodromal of the onset of solid tumors in a healthy subject. The method includes an evaluation cycle of a genetic stability or instability condition and at least one repetition of the evaluation cycle. The repetition cycles are performed periodically on the subject, with the frequency depending on the result of the previous cycle. Each cycle includes taking a sample, verifying the presence of mutations, verifying the frequency of mutations, recording the mutations, defining or updating a genetic instability index of the subject, evaluating, in each repetition cycle, the subject's entry into a prodromal genetic condition upon the onset of one or more solid tumors or groups of solid tumors on the basis of a threshold value (I_{TS},I_{GS}) of the genetic instability index (I_{T},I_{G}), defined for each single gene or group of genes, being exceeded.

### CROSS-REFERENCE TO RELATED APPLICATIONS

This application is a divisional of application Ser. No. 16/607,993 which is the national stage of international application number PCT/IB2017/054231 filed Jul. 13, 2017, hereby incorporated by reference.

### TECHNICAL FIELD

The present invention generally refers to the field of cancer prevention and more in detail to solid tumors prevention.

In particular, the invention includes a method for searching and identifying, in an individual, a genetic condition that is prodromal of the onset of solid tumors, and the statistical prediction of the presence of significant possibility of contracting such tumors in individuals considered at risk.

### BACKGROUND ART

A solid tumor is made up of one or more masses of tissue consisting of tumor cells and stroma (in turn composed of different types of cells and extracellular matrix), characterized by an abnormal and uncontrolled growth, which in some cases can cause a systemic pathology.

The tumors are pathologies caused by genetic alterations; they develop in different steps, consecutive in time, in which a series of subsequent mutations accumulate in the genetic heritage of one or more cells.

A genetic instability condition, associated to an increase of the genetic alterations of the subject is a marker of particular risk for tumor formation.These genetic alterations increase the risk for the carrier cells to cause tumor cells lines.

This process has been thoroughly studied for the colorectal cancers. In the genesis of these tumors, the first mutation, that involves genes called gatekeepers/caretakers, responsible for the control of the genetic stability, causes a selective advantage in the growth of a normal epithelial cell, allowing it to dominate the surrounding cells and become a microscopic clone.

The best known of the gatekeeper genes in the colon is the APC gene.

Almost all (about 80%) the colorectal tumors are characterized by a mutation of the APC gene. The small adenoma caused by this mutation grows very slowly, however, if a second mutation occurs in another gene, as for example the KRAS gene (or ATK1, etc.), this triggers a new phase of clonal growth which causes the expansion of the number of cells involved. The cells with only the APC mutation can remain in the adenoma, but their number is limited with respect to those with mutations in both (or more) genes.

This process of successive mutations, followed by corresponding clonal expansions, continues with mutations in other genes, such as PIK3CA, SMAD4 and TP53, with high probability to generate a malignant neoplasm, which can extend through the underlying basal membrane and metastasize, first generally toward the regional lymph nodes and then to distant organs, such as liver or lungs.

During the last decade, a thorough series of sequencing disclosed the genomic panorama of the most common forms of human cancer. Sequencing of the genome of many tumors has given information related to thousands of mutations and other genomic alterations. At present, more than ten thousand tumor genomes have been sequenced and many other tumor genomes will be characterized in the near future, with gradual reduction of the sequencing costs.

Sequencing of the tumor genomes has allowed different “genes with driver mutations” to be classified and will allow classification of many others. A driver mutation is a mutation within a gene which provides a significant advantage in the growth.

Up to now, the carried out studies have allowed identification of about 140 genes, which, when mutated, can facilitate or “drive” the oncogenesis. A typical tumor can generally contain two to eight such mutations in “driver genes”. The remaining mutations are to be considered transitory and they do not offer any advantage of cell growth.

The driver genes are often involved in the regulation of the key molecular pathways of the cell, which regulate, in different forms and modes, three main cellular processes: the cellular differentiation, survival (through pro- or anti-apoptotic signals) and maintenance. At present, one of the most pressing needs in the basic cancer research is a deep understanding of such different pathways. However, even the degree of understanding reached so far in the structure of the tumor genomes is sufficient to guide some current therapeutic choices and has determined the development of more effective approaches in the reduction of cancer morbidity and mortality.

The screening and early diagnosis programmes play an important role in improving healthy survival and reducing mortality in cancer patients. Since non invasive approaches for early diagnosis contribute to encourage the patient's cooperation, it is definitely advisable to include them in screening and prevention programmes.

The increasing knowledge of the molecular pathogenesis of the tumor pathologies and the rapid development of new techniques of molecular analysis are contributing to the development and achievement of identification and analysis of the early molecular alterations in the body fluids. Extracellular DNA (“cell-free DNA” or “cfDNA”) can be found in the serum, plasma, urine and other body fluids. Therefore, sampling and molecular analysis of the cfDNA represents a kind of “liquid biopsy” which can constitute a kind of “circulating image” of various specific pathologies.

In the blood, the apoptosis seems to be the most frequent process that generates cfDNA, although in the cancer patients, the portion provided by necrotic processes cannot be completely neglected. An interesting study analyzing cfDNA from the plasma of 32 patients affected with stage 4 colorectal cancer has shown that in 34.4% of the patients the taken DNA had two magnitude dimensions (166bp and 332bp).

Stroun et al. have described that different cancer alterations can be identified in the cfDNA of a patient. Various articles published afterwards have confirmed that the cfDNA contains specific alterations correlated to the presence of tumors, such as mutations, methylations and variations of the number of copies (“copy number variations” or CNVs) of specific genes, directly referable to tumor cells. thus confirming the existence of the circulating tumor DNA (ctDNA).

Various studies, aimed at correlating the selected samples of tissue and plasma, have been carried out in order to confirm that the analysis of the circulating cfDNA can be used as diagnostic instrument.

An evaluation with NGS techniques (next-generation sequencing) performed on 50 tumor genes, that covers 2,800 COSMIC mutations (Catalogue Of Somatic Mutations In Cancer- http://cancer.sanger.ac.uk/cosmic) in 60 tumor tissues and 31 plasma samples from 17 patients with metastatic breast tumor has revealed a 76% concordance between tissue and plasma. From these data the authors have drawn a conclusion that plasma can be considered the biological sample adopted for the screening of tumors as a substitute of the metastatic biopsy.

The above mentioned results have been confirmed in a group of 34 patients suffering from 18 different types of tumor: the analysis has involved 46 genes and covered 6,800 COSMIC mutations in samples of tissue and plasma. Twenty seven out of thirty four patients have shown a 97% agreement between the mutations found in the tissue samples and those found in the ctDNA. Consequently, the ctDNA-based NGS analysis could revolutionize the management of patients suffering from cancer pathologies which are potentially curable or metastatic.

Document US 2013/143747 describes methods for a molecular classification of diseases and particularly molecular markers for cancer. The methods include analysis of panels of genes obtained from a blood sample. Synthesized mRNA or cDNA are obtained from the sample and the mutational status of the genes of the panels are determined. The presence of a particular mutational status in particular genes in a certain panel indicates that (a) the patient had a cancer and/or (b) the patient has that particular cancer. By the methods described in US 2013/143747 a screening is carried out with the purpose of finding people that already have a cancer, and even people that have a particular cancer. The patient has already developed the disease, even at an advanced step. US 2013/143747 does not provide that a genetic instability index can also be defined on the basis of an increase in the frequency of mutations with respect to one or more previous evaluation cycles Nor this document suggests that a predetermined set of genes and selected mutations can be defined in view of the subject's anamnesis. According to the methods of US 2013/143747, the type of genes in which mutations occur determine which tumor has been found, and an actual and real diagnosis of an ongoing pathological process is obtained. Not a genetic condition prodromal of the onset of solid tumors in a healthy subject.

Document US 2016/0102358 discloses a method of assessing an individual subject's risk of developing different types of cancer by identifying significant statistical association between multiple genetic markers and cancer risk for a variety of different cancers. In particular, the invention disclosed in US 2016/0102358 provides a method of producing a personalized cancer risk report for a subject by determining from a nucleic acid sample of the subject a genotype at a plurality of biallelic polymorphic loci. The risk is calculated based on the genotype determined for each plurality of biallelic polymorphic loci.

An article of GAGAN et Al, in “GENOME MEDICINE”, Vol. 7, No. 1, 29 July 2015, pages 80-89, discloses methods for diagnosing and managing a patient having, for example, cytophenia. The methods include obtaining a sell-free DNA sample from peripheral blood plasma or serum of the patient, performing a mutation analysis of the of the cell free DNA sample on a panel of selected genes, and administering to the patient a treatment for management of a hematologic malignancy.

Also the methods disclosed by GAGAN et Al. are directed to treatment of an already developed disease and does not teach nor suggest detecting a condition prodromal of the onset of solid tumors in a healthy subject.

The article of DE MATTOS-ARRUDA LETICIA et Al, in MOLECULAR ONCOLOGY, Vol. 10, No. 3, 17 Dec. 2015, pages 464-474, discloses a diagnosis of cancer based on analysis of liquid biopsy while the article of MARK SAUSEN et Al., in NATURE COMMUNICATIONS, Vol. 6, No. 7686, 7 Jul. 2015, pages 1-6, discloses a method to non-invasively scan tumor genomes and quantify tumor burden. Also in these documents the described methods are directed to treatment of an already developed disease and no teachings or suggestions are given for detecting a condition prodromal of the onset of solid tumors in a healthy subject.

Document US 2016/0130648 discloses methods for treating, managing, diagnosing and monitoring myelodysplastic syndrome and other hematologic malignancies. These methods comprise sequencing analysis conducted on cell-free DNA from peripheral blood plasma or serum. By the methods described in US 2016/0130648 diagnosis and post-diagnosis treatments are carried out with the purpose of finding and treating people that already have a cancer. No teachings or suggestions are given for detecting a condition prodromal of the onset of solid tumors in a healthy subject.

### Technical Problem

The evaluation of the risk status constitutes an essential component of the testing procedures and genetic analysis. There is a strong likelihood that in the very near future this type of approach will be implemented in a systematic manner in the prior assessment of the cancer pathologies.

On the other hand, it is more and more frequent that “healthy” persons ask to have genetic tests for predisposition to serious pathologies or detecting a pre-pathological status. Also for this reason the physicians should introduce techniques for the definition and management of risk, and for identifying surely or very probably pre-pathological conditions, in their routine monitoring programmes.

The genetic risk refers to the likelihood of an individual carrier of a mutation associated specifically to a determined pathology—in particular as regards the present disclosure, a cancer pathology—actually developing this pathology. On the other hand, the identification of a pre-pathological condition or a condition prodromal of the onset of a pathology relates to the detection of biomolecular signals indicative of a genetic instability situation, which, due to a subsequent evolution, can cause over time, certainly or very likely, the onset of the pathology.

Given that, as already pointed out several times in the text, the carcinogenesis is influenced by both environmental factors and hereditary predisposition, the genetic background associated with a specific disorder considerably affects the definition of the risk correlated to this particular disorder.

According to Wang E. et al. an algorithm based on a series (network) of characteristic elements can be adopted to generate a genetic model of the key components of the tumor and to connect mutating genotypes with clinical phenotypes. The use of this pattern (and others similar) illustrates the strategies for the prediction of possible tumor therapeutic targets, probability of recurrence and risk of the onset of the tumor on the basis of an individual profile of the patient's genomic sequence.

To sum up, prediction methods deriving from a model based on a network of characteristic elements can be used in the diagnosis and optimized management and prevention of the cancer pathologies programmes.

Genomic instability arises in normal cells through accumulation of genetic and epigenetic changes and has been shown to occur over a variable time span, ranging from years to decades (Gerlinger et al., 2012; Lengauer, Kinzler, & Vogelstein, 1998; Yates et al., 2015; Zhang et al., 2014).

The vast majority of normal cells that have acquired genomic instability features are thought to be cleared away by the immune system, while a minimal fraction might eventually progress and give rise to cancer (Nakad & Schumacher, 2016).

Upon development of cancer, specific genomic alterations can be identified and used to provide the rationale for specific treatment options. Monitoring genomic instability could hence be crucial to identify early mutational events that are associated with higher risk of developing cancer, but it is largely unfeasible using traditional tissue-based approaches. In contrast, liquid biopsy might offer the possibility of detecting early genomic aberrations and understanding cancer cells evolution, allowing the interrogation of cancer cells longitudinally and in a minimally invasive fashion. Liquid biopsy is a broad term that refers to the sampling of non-solid biological tissue, most commonly blood, but also saliva, urine and other body fluids. Interestingly, cancer patients typically present higher levels of total circulating cfDNA (cell-free DNA) compared to healthy individuals ((Alix-Panabieres & Pantel, 2016; Bettegowda et al., 2014).

The majority of DNA fragments in circulation, including cfDNA and ctDNA (circulating-tumor DNA), measures between 180 and 200 nucleotides in size. This observation suggests that cell death associated to physiological tissue remodeling events is likely to produce the majority of DNA fragments found in the bloodstream. Conversely, ctDNA is derived from tumor cells undergoing either apoptosis or necrosis and represents only a minor fraction (<0.1-10%) of the total circulating cfDNA. A typical output for cf/ctDNA analysis is generated through the evaluation of genomic events in well-defined cancer-associated genes, often by using highly sensitive technologies as digital PCR or next-generation sequencing (NGS) {Goodwin:2016fy}. ctDNA analysis has been applied to monitor response to therapy, to detect minimal residual disease and to assess the development of resistance to therapy (Diaz et al., 2012; Siravegna et al., 2015; Tokudome & Hayes, 2013).

Currently, the most common clinical use of liquid biopsy is monitoring tumor burden in response to therapy and the identification of resistance-associated genetic alterations to inform treatment decision ((Azad et al., 2015; Fribbens et al., 2016; Misale et al., 2012; Weigelt et al., 2017).

The introduction of molecular barcodes has considerably enhanced the sensitivity of sequencing methods (down to 0.01% of variant allelic frequency), at the price of additional costs given the extremely high level of sequencing depth required (i.e., ˜25,000 coverage) ((Kinde, Wu, Papadopoulos, Kinzler, & Vogelstein, 2011; Newman et al., 2016; Schmitt et al., 2012).

Taking advantage of this and further technological advances, a number of studies have described clinically-relevant genetic alterations in patients with early-stage cancers, even at a sensitivity of <1 mutant template molecules per milliliter of plasma (Abbosh et al., 2017; Aravanis, Lee, & Klausner, 2017; Bettegowda et al., 2014; Cohen et al., 2017; 2018)

Nevertheless, among the potential clinical applications of ctDNA analysis, early detection remains the most ambitious, given the substantial number of patients and healthy controls necessary to establish the required sensitivity and specificity for such testing approach (De Mattos-Arruda et al., 2014).

Furthermore, age-related clonal hematopoiesis in asymptomatic patients might be as well a source of false positive results (Cohen et al., 2018), a phenomenon not entirely understood in the general population but well-established in patients that survived (i.e. considered clinically cured) hematological cancers (Cree et al., 2017; Genovese et al., 2014).

Importantly, monitoring overall genomic instability in healthy individuals could lead to the identification of subpopulations that might enter into early access screening prevention programs. Nevertheless, the recovery and characterization of cfDNA in healthy individuals might prove challenging, given that cfDNA is less abundant in these subjects, and that there are only few studies describing cfDNA analysis in healthy controls (Cohen et al., 2018; Cree et al., 2017). It is intuitive, therefore, that the above-described model could only be successful after an adequate technical validation, proving that extraction and analysis of cfDNA isolated from healthy individuals is technically achievable. To this end in our proof of principle study, we investigated the possibility to interrogate cfDNA in a collection of clinically healthy individuals (i.e. not affected by any manifest medical condition) at the time of blood collection. First, we evaluated the reliability of our testing strategy by analyzing cfDNA samples obtained from patients with an histologically confirmed diagnosis of breast or lung cancer. Specifically, we assessed the mutational status concordance in matched tissue and plasma specimens. We then analyzed a group of healthy donors comprising both individuals that did not develop any tumor within 1 to 10 years (average=8.39 years, Table 1) of follow-up as well as individuals that developed either a benign neoplasm or cancer during the follow-up time. Altogether, our study demonstrates the technical feasibility of extracting and analyzing cfDNA in healthy individuals to study genomic alterations, by means of molecular barcoded ultra-deep sequencing.

### Objects of the Invention

The main object of the invention is to provide a monitoring method capable of obtaining an evaluation, for a clinically healthy individual, of his/her entry into a genetic condition prodromal of the onset of one form of solid tumor, that is a condition that, sooner or later, almost certainly will make the individual develop the form of tumor being monitored.

### SUMMARY OF THE INVENTION

This and other objects are obtained by the invention, which relates to a method (based on an algorithm and an associated database) for searching and identifying, in a healthy individual, a genetic condition that is prodromal of development of solid tumors, with the exception of the brain tumors. This method defines the above mentioned condition on the basis of a series of periodical evaluation cycles about the mutation frequency that involve a panel of genes chosen from those associated to the onset of the cited solid tumors.

The method includes taking of a biological sample from an individual (constituted by body fluids, such as blood, urine or spinal fluid), isolating and amplification of the DNA and sequencing thereof. A subsequent analysis step includes evaluation of the presence of one or more mutations that involve the cited panel of genes, chosen from a list of known mutations for the monitored genes and indicative of an evolution toward the formation of neoplastic cells.

In particular, an evaluation with NGS techniques (next-generation sequencing) is performed on 50 genes, and a total of 2,800 mutations classified in the COSMIC database (Catalogue Of Somatic Mutations In Cancer—http://cancersanger.ac.uk/cosmic).

Every evaluation cycle detects, for every monitored gene, the existence of mutations involving it, with particular attention to mutations which occur in known hotspots (positions frequently mutated in cancer patients). In the case of a mutation, it is checked if this detected mutation has already been found in previous evaluation cycles and at which level (mutated percentage or fraction—allelic frequency). In negative case, the new mutation involving this gene is registered and an algorithm calculates a value indicative of the trend that the frequency of mutation shows over time. This value constitutes the “Key Risk Indicator” of the system. The trend is represented with a numerical value, obtained by calculating a relation between the last value of the mutation frequency and the values obtained in the previous evaluation cycles, and its tendency can be represented in a diagram to provide indications of the level of the genetic stability or instability.

Once it has been detected that the threshold level defined for the frequency of mutation is exceeded, according to the identification method of the invention, a report is issued about the entry in a genetic instability path, during which further mutations will make the individual develop a solid tumor over time, certainly or very likely.

The panels of genes and their mutations taken into consideration for every evaluation cycle can include again the whole range of 50 genes associated with the onset of solid tumors, or only some genes and hotspots associated only with one or more chosen tumors.

In particular, it is possible to monitor the individual's genetic situation, whose instability is associated with the onset of only one type of tumor or a single family of tumors. In this case, the number of genes being analysed is limited to those directly associated with that tumor or that group of tumors.

According to the invention, the panel of genes and the mutations to be analysed can be chosen on the basis of the subject's anamnesis, obtained by a historic-family survey.

When the detected frequency of mutation shows a growth trend that exceeds a predefined value, for example 10%, (value which can be updated anyway, depending on the data that will be accumulated over the years) that is an increased number of the allelic frequency (according to what has been defined above) for a given mutation (for example, the allelic frequency of the mutation of the APC gene passes from of 0.1% to 5% in a subsequent test repetition), the monitoring system increases the sensitivity related to the panel being examined (i.e. it will ask the patient to carry out the test again, this time using a different panel having a greater sensitivity and analytical specificity with respect to the test of the first level) with regard to the genes involved in the increase of the mutations, up to 100%.

The method includes a continuous update of the evaluation parameters, such as the repetition frequency of the analysis cycles of the chosen panel of genes and the sensitivity related to the genes being analysed. In particular, the repetition frequency is defined on the basis of the stability index; more precisely, higher values of the instability index correspond to a greater repetition frequency of the tests, since it is assumed that an instability situation tends naturally to grow, and causes a higher probability to have new significant mutations more rapidly.

According to the invention, all the raw data and obtained results, related to the DNA analysed for each evaluation cycle, are recorded and processed during the subsequent cycles to improve the accuracy of evaluation as the quantity of the available data increases.

In order to make evaluation and prediction of the risk status as little invasive as possible, the biological sample taken from the individual to isolate the DNA to be analysed is constituted by a liquid biopsy (as already defined herein). In a preferred embodiment of the invention, the liquid biopsy is a peripheral blood sample.

In an embodiment of the invention, the liquid biopsy is urine.

The DNA isolated from the biological sample and used for the evaluation and prediction of the risk status is cfDNA (cell free DNA).

In an embodiment of the invention the cfDNA is isolated from plasma separated from the peripheral blood sample.

In another embodiment of the invention the cfDNA is isolated from plasma separated from the peripheral blood sample.

The method according to the invention includes also searching ctDNA (circulating tumor DNA) in the collected liquid biopsy.

When the ctDNA is detected, the method for evaluation and prediction of the risk substantially stops performing its task, since it means that at least one of mutation lines has caused the formation of neoplastic cells. At this point, the system for risk evaluation and prediction issues information aimed at transferring the control of the individual to an early detection system, such as, for example, the SCED system—Solid Cancer Early Detection, developed and used by the present Applicant.

### BRIEF DESCRIPTION OF THE DRAWINGS

The characteristics of the invention, as they become evident from the claims, are pointed out in the following detailed description, with reference to the enclosed tables of drawings, in which:

FIG. 1 illustrates a flow chart of the method for searching and identifying a prodromal genetic condition at the onset of solid tumors according to a general embodiment of the invention;

FIG. 2 illustrates a list of the panel of genes involved in the evaluation of the mutations which define a genetic stability or instability condition according to the method of the invention.

FIGS. 3A, 3B, and 3C show total cfDNA yield of plasma samples deriving from healthy donors or cancer patients.

FIGS. 4A, 4B, 4C, 4D, 4E, 4F, and 4G show a comparison of preanalytical variables from healthy and cancer donor samples.

FIG. 5A, 5B, 5C, and 5D show a concordance analysis of liquid and tissue biopsy in cancer patients.

FIGS. 5A, 5B, 5C, 5D and 5E show genetic alterations detected in the cfDNA of healthy individuals.

FIGS. 7A, 7B, and 7C show cfDNA size distribution in healthy individuals and cancer patients.

FIGS. 8A, 8B, and 8C show a concordance analysis of matched plasma and tissue samples from breast and lung cancer patients:

### DETAILED DESCRIPTION OF EMBODIMENTS

The present invention relates to a method for searching and identifying a genetic condition prodromal of the onset of solid tumors in a healthy individual, with the exception of brain tumors, for monitoring the individual over time in relation to possible entry in said condition, on the basis of the evolution of the trend of the individual's genetic stability.

Brain tumors are generally excluded from the approach used in the method for searching and identifying a genetic condition prodromal of the onset of solid tumors according to the invention. At present, scientific evidence in not enough to allow this approach to be used also for the brain tumors. The set of genes associated with the onset of this type of tumors has not been properly identified yet, and at present, they seem to be associated mainly with the DNA methylation state rather than with a specific mutation of its sequence. In fact, the proposed panels are not fit for evaluation of the DNA methylation state. Moreover, it is necessary to specify that, as already described by Bettegowda, C. et al. (Bettegowda, C. et al. Detection of circulating tumor DNA in early- and late-stage human malignancies. Science translational medicine 6, 224ra224, doi:10.1126/scitranslmed.3007094-2014), very likely the blood-brain barrier forms a filter that reduces considerably the presence of cfDNA in the general circulation. Therefore, the present techniques of extraction and subsequent analysis do not allow providing data strong enough to be used for an analysis of tumor risk.

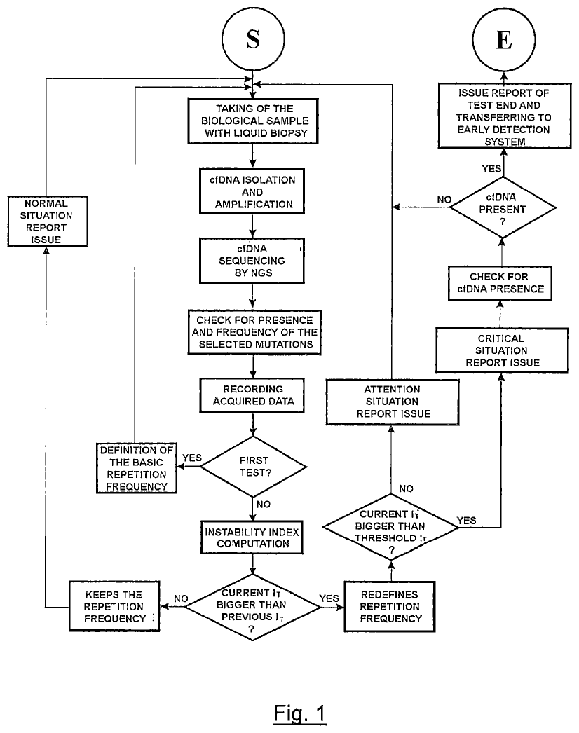

The method includes carrying out, at predefined intervals, a series of periodical evaluation cycles of the mutation frequency that involve a panel of genes chosen from those associated to the onset of the cited solid tumors. FIG. 1 illustrates, by way of example, a possible workflow of the steps of the method that will be described later on. Non fundamental changes of the workflow are possible without departing from the scope of the invention.

Unless otherwise stated, it is agreed that the technical terms used in the present treatment have a meaning commonly and unambiguously known to persons of ordinary skill in the field (for example, “liquid biopsy”, “DNA isolation”, “DNA amplification”, “DNA sequencing”, “ctDNA”, “cfDNA”, “Circulating Tumor Cells”, etc.). It is also assumed that the techniques of molecular biology and genetic engineering, which are referred to and which are intended to be used for carrying out the method according to the invention (for example, “NGS—Next Generation Sequencing”), are standard techniques commonly used in practice and are also well known to the persons of ordinary skill in the field.

For each cycle of search and identification of a genetic condition prodromal of the onset of solid tumors, the method proposed by the invention includes taking of a biological sample from an individual, isolating of the DNA from the biological sample and then sequencing thereof, preferably with the NGS technique, after having suitably amplified the relevant fraction of DNA.

In order to make the evaluation and prediction of the risk status as little invasive as possible, the biological sample taken from the individual to isolate the DNA to analyse consists of a liquid biopsy. A liquid biopsy is substantially formed by a liquid or semi-liquid biological material circulating in the individual and produced by him/her by secretion or excretion substantially of a body fluid.

In a preferred embodiment of the invention, the liquid biopsy is a peripheral blood sample, which is taken and treated, if necessary, in order to separate the plasma or serum, depending on the subsequent use.

In a different embodiment of the invention, the liquid biopsy is urine.

According to the invention, the DNA isolated from the biological sample and used for the evaluation and prediction of the risk status is cfDNA (cell free DNA).

The existence of circulating free DNA, cfDNA (Cell Free DNA) has been demonstrated for the first time by Mendel and Metis about 70 years ago. The above mentioned DNA derives from the necrotic cells (premature death) and/or apoptotic cells (programmed death) and is generally released by all the types of cells. About 40 years after the discovery of the cf-DNA, Stroun et al. have demonstrated that specific carcinogenic alterations could be identified also in the cf-DNA. Afterwards, several articles have been published that confirm the existence of the circulating tumor DNA (ctDNA) by studying specific alterations associated with tumors. The ctDNA is thus a portion of the total cfDNA and has been estimated to represent 0.01% to 1% in very early stages up to 40% in the advanced stages, as already described by Bettegowda, C. et al. (Bettegowda, C. et al. Detection of circulating tumor DNA in early- and late-stage human malignancies. Science translational medicine 6, 224ra224, doi :10.1126/scitranslmed.3007094-2014).

As already mentioned, in the blood, the apoptosis seems to be the most frequent process that generates cfDNA, although the portion provided by necrotic processes in cancer patients is not totally neglectable.

The quantity and variability of cfDNA in serum and plasma seems to be considerably greater in ill patients rather that in healthy controls, especially in cases of an advanced stage tumor rather than in early stages.

It is to be considered that the quantity of the cfDNA is influenced also by physiopathological conditions such as an inflammatory process, and since the cfDNA has a very low stability (from 15 minutes up to several hours), thus the reliability and coherence of the result are not always assured. In this case, an approach based on the periodical repetition of the test offers the advantage of obtaining a lower incidence of false negatives.

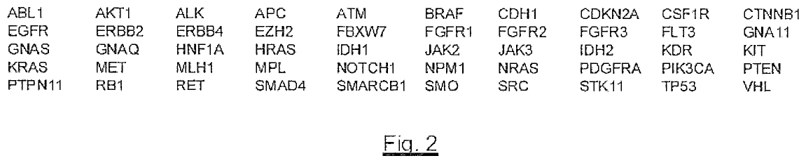

A study with an NGS panel, which can evaluate 50 genes involved in cancer (see FIG. 2), covering 2,800 mutations (COSMIC) in 60 solid tumors and 31 blood tumors, has analysed 17 patients with breast metastatic tumor and a 76% concordance between tissue and plasma has been estimated. From these data the authors have drawn a conclusion that plasma can be considered the biological sample adopted for the screening of tumors as a substitute of the metastatic biopsy.

In an embodiment of the invention the cfDNA is isolated from plasma separated from the peripheral blood sample taken from the individual.

In a different embodiment of the invention the cfDNA is isolated from serum separated from the peripheral blood sample taken from the individual.

According to known techniques, the cfDNA present in the plasma can be isolated using magnetic beads covered with silica or silicone resins. In both cases, the capacity of DNA (negatively charged) to bind with silica (positively charged) in the presence of high concentrations of chaotropic salts having pH near 7.5, is exploited (Chen and Thomas, 1980; Marko et al. 1982; Boom et al. 1990). The binding of DNA to silica is induced by the dehydration induced by chaotropic salts and by the formation of hydrogen bonds which compete with weak electrostatic repulsions (Melzak et al. 1996). Afterwards, the exceeding salts, proteins, carbohydrates, metabolites and other contaminating substances are removed by repeated washing with alcoholic solutions. Finally, the purified DNA is eluted by a low ionic strength solution (like TE-buffer or water).

The next step of the method for evaluation and prediction of the risk according to the invention consists of the analysis of the chosen panel of genes (FIG. 1), with particular attention to a chosen group of hotspots, which includes the evaluation of the presence of one or more mutations that involve the cited panel of genes, chosen from a list of known mutations for the monitored genes and indicative of an evolution toward the formation of neoplastic cells.

For example, about 10% of the patients suffering from lung tumor (non small cell type) in the United States and Europe are characterized, from the genetic point of view, by the mutations of the EGFR gene (Lynch et al 2004 ;. Paez et al 2004 ;. Pao et al., 2004). These mutations take place mainly within the EGFR exons 18-21, which codify a portion of the tyrosine kinase domain of EGFR. About 90% of these mutations involving the exon 19, are delections or in the exon 21 are SNV (like the mutation L858R) (Ladanyi and Pao 2008). These mutations increase the tyrosine kinase activity of the EGFR protein, which determines a hyper-activation of the cellular pathways that stimulate the survival of the tumor cells (Sordella et al. 2004). Regardless of the ethnic origin, mutations of the EGFR gene are more frequently found in non smoking female patients (less than 100 cigarettes during the patient's whole life) with adenocarcinoma type histology (Lynch et al. 2004). However, these mutations that involve the EGFR gene can be found also in other subgroups of patients with lung carcinoma, thus also in smokers. The presence of the above mentioned mutations in the cfDNA of a seemingly “healthy” patient represents an obvious alarm bell which must be necessarily followed by a monitoring and a series of thorough exams to evaluate the presence of a tumor mass.

In particular, cfDNA is evaluated with NGS techniques (next-generation sequencing) on 50 genes, a list of which is provided in FIG. 2, which covers currently 2,800 COSMIC mutations (Catalogue Of Somatic Mutations In Cancer).

It will be appreciated that, for the purposes of the invention, both the list of genes and the list of the mutations which are evaluated, must not be considered static, since the intensive research activity in this field as well as the available modern sequencing and analysis techniques can lead to the identification of new genes, new hotspots or new mutations involved in the carcinogenesis of one or more solid tumor forms.

Each evaluation cycle searches, for each monitored gene, the mutations involving it (see the above example of the EGFR), with particular attention to mutations that occur in known hotspots. When a significant mutation is present, this is recorded in a memory area of a computerized system which is physically appointed to carry out computational steps of the method (FIG. 1).

In each repetition cycle of the test, that is in each cycle after the first one, it is checked whether the detected mutation has already been detected in the previous evaluation cycles. In a negative case, the new mutation involving this gene is registered and an algorithm calculates a value indicative of the trend that the frequency of mutation shows over time. This value constitutes the “Key Risk Indicator” of the system.

The trend is represented by a numerical value, obtained by the computation of a relation between the last frequency value of the mutation and the values obtained in the previous evaluation cycles, and its tendency can be represented in a diagram to provide indications of the level of the genetic stability or instability.

In a non-limiting embodiment of the method according to the invention, the comparison between the two checks can be summed up by an overall genetic instability index IT according to the formula:

I_{T}=

Where:

T is the number of genes on which the control is performed

It is to be noted that in this case the overall instability index I_{T} expresses the overall situation, without distinguishing between the variations on the specific genes.

In a similar way, it is possible to define an instability index I_{G} of a specific gene according to the formula:

$${I}_{G} = {\sum }_{j = 1}^{n} \frac{Δ {F}_{j}}{n} \#$$

Where number of evaluated hotspots of the gene on which the control is performed #

The panels of genes and their mutations taken into consideration for every evaluation cycle can include again the whole range of 50 genes associated with the onset of solid tumors (FIG. 2), or only some genes and hotspots associated only with one or more chosen tumors.

In particular, it is possible to monitor the risk of the onset of only one type of tumor, or a single family of tumors. In this case, the number of genes being analysed is limited to those directly associated with that tumor or that group of tumors.

In any case, the definition of the instability index according to the above described process can involve, depending on the prefixed search target, a single gene, a panel consisting of a set of genes associated to the onset of a specific tumor or family of tumors, or the whole panel consisting of the 50 genes (at present, or more genes, if, in future, other genes are identified) associated with the onset of solid tumors in general.

According to the invention, the panel of genes and the mutations to be analysed can be chosen on the basis of the subject's anamnesis, obtained by a historic-family survey.

When an evaluation cycle detects a frequency of mutation that expresses a growth trend higher than 10%, (value which can be updated depending on the data that will be accumulated over the years) that is an increased number of the allelic frequency for a determined mutation (for example, the allelic frequency of the mutation of the APC gene passes from of 0.1% to 5% in a subsequent test repetition), the monitoring system increases the sensitivity related to the panel being examined (i.e. it will ask the patient to carry out the test again, this time using a different panel having a greater sensitivity and analytical specificity with respect to the test of the first level) with regard to the genes involved in the increase of the mutations, up to 100%.

For example, using the panels and commercial technology “Oncomine® cfDNA”, extremely low detection levels are reached, equal to 0.05%. It means that the system, based on a particular chemistry (“Oncomine® TagSequencing”), different from the screening of the first level, is capable of identifying a mutation present in barely 0.05% of the analysed DNA sample.

The method includes a continuous update of the evaluation parameters, such as the repetition frequency of the analysis cycles of a chosen panel of genes and the sensitivity related to the genes being analysed. In particular, the repetition frequency is defined on the basis of the instability index (I_{T} or I_{G}, depending on whether a panel of several genes or a single gene is analysed); more precisely, low values of the instability index correspond to a basic repetition frequency of the tests, which can be for example of one test per year. Even moderate increase of such index value can suggest an increased repetition frequency, since it is considered that an instability situation tends naturally to increase and implies a bigger probability of new significant mutations in shorter time. The exceeding of a specific threshold value I_{TS}, I_{GS} of the instability index I_{T}, I_{G} identifies an evolution of the sequence of mutations in a prodromal genetic condition at the onset of the tumor or group of tumors being monitored, that is in a path which will lead the individual, certainly or very likely, to develop the above mentioned tumor, or in any case, at least one of the monitored tumors. The threshold value of the instability index can be for example set to 0.1.

According to the invention, gradual increase of the repetition frequency of the tests allows best monitoring of the individual's situation and understanding when the specific mutations can occur, indicative of an oncogenesis underway.

According to the invention, all the raw data and obtained results, related to the DNA analysed for each evaluation cycle, are recorded and revised during the subsequent cycles to improve the accuracy of evaluation as the quantity of the available data increases.

According to another characteristic of the method of the invention, the sequencing of the cfDNA for the study of the somatic mutations is combined with the analysis of the germ mutations. For this purpose, according to what has been already described, the cfDNA with the Hotspot Cancer Panel (HSCP) is isolated and sequenced, which allows to study 2800 mutations in 50 genes involved in neoplastic processes with a 1% sensitivity.

Moreover, the individual's germ DNA, for example lymphocyte DNA, is isolated and sequenced, and the presence of the same mutations in the above mentioned DNA is checked., For the purposes of the invention, the individual's lymphocyte DNA can derive indifferently from the same liquid biopsy from which the cfDNA has been taken and isolated, or from another, recent or even much older biopsy, since the germ mutations present in the lymphocyte DNA form part of the individual's genetic heritage.

The lymphocyte DNA is sequenced preferably with the same reading degree of the cfDNA sequencing to obtain directly comparable results.

A mutation is thus defined somatic, if it is present in the cfDNA analysis and not in the lymphocyte DNA sequenced with the same reading degree. Thus the following sets of mutations are defined:
Set A: consisting of mutations found in the cfDNA;
Set B: consisting of mutations found in the lymphocyte DNA analyzed by the same panel used for the cfDNA;
Set C: consisting of somatic mutations defined as the mutations present in the cfDNA, but not in the lymphocytes.

When a somatic mutation is identified (that is, in the analysis, the set C is different from empty) the tissues in which such a somatic mutation has been mostly found, are evaluated with known operational techniques, by the COSMIC database.

The next step is to study if there is a higher probability to develop a tumor of these tissues, from the analysis of the germ line, using operational techniques, known also in this case. The above mentioned approach will be carried out by a customized panel, whose definition is a function of the found somatic mutation or somatic mutations, and allows to evaluate the individual's susceptibility to particular tumors on the basis of the mutated gene (or the mutated genes) in the cfDNA.

Furthermore, if the somatic mutation concerned involves the lung, colon or breast, the circulating DNA is analysed with a higher sensitivity (up to 0.1%), for example by means of commercial panels Oncomine®, and using the commercial technology Tag_Seq®.

When these first analyses are completed, the evaluation is finished proposing an oncological consultation to explain and evaluate the results and to plan new tests in order to monitor the individual being examined.

The method according to the invention includes also searching the ctDNA (circulating tumor DNA) in the taken liquid biopsy, as an additional activity that completes the evaluation of the genetic stability and identification of the prodromal phase of the oncogenesis. Such operation can be performed only when the calculated instability index exceeds the prefixed threshold value I_{TS}, I_{GS}, as indicated in FIG. 1, or also when the values of the instability index are below this threshold, as a precaution.

When the ctDNA is detected, the method for evaluation of the genetic stability and identification of the prodromal phase of the oncogenesis substantially ends its function, since it means that at least one of the mutation lines has caused the formation of neoplastic cells. At this point, the system for evaluation of the genetic condition gives information to transfer the control of the individual to an early detection system, (“Early Detection”), such as for example the “SCED system—Solid Cancer Early Detection”, developed and used by the present Applicant.

Some applications will be described in the following by way of example, focused on the evaluation of the genetic stability and identification of the prodromal phase of the oncogenesis related to single solid tumors or families of solid tumors, and in particular to lung, breast and ovarian, and colorectal tumors.

### EXAMPLE 1

### Lung Tumors

In an embodiment of the invention, the method for the evaluation of the genetic stability and identification of the prodromal phase of the oncogenesis is applied to monitoring of the genetic condition associated with the onset of the lung tumors.

The most serious risk factor for the onset of a lung tumor is represented by cigarette smoking. A clear correspondence between the amount of smoke inhaled by a smoker and the increase of the probability to contract such tumor has been widely proved and is already considered a fact.

Several studies report that the risk to contract a lung tumor is 14 times higher for smokers than non smokers (up to 20 times for heavy smokers—more than 20 cigarettes a day). The cigarette smoke is responsible for 8/9 out of every 10 lung tumors, though atmospheric pollution, family predisposition for this type of cancer, and the presence of other lung diseases may increase the likelihood of contracting a tumor.

On the basis of the quantification of his/her personal risk that specific mutations of particular genes associated with lung tumors and the number and frequency of such mutations can generate tumor cells in future, the person being monitored is offered the possibility of knowing, with adequate reliability, whether a development is detected and which evolution stage has been reached.

According to the present method, the definition of the prodromal state of the tumor onset in this case is connected to the mutation of 11 genes involved directly in lung tumors, in particular of 169 different hotspots. Table 1 provides a list of genes and hotspots that compose the panel, which will be evaluated.

An application of the method for the evaluation of the genetic instability with regard to the panel related to lung tumors for a healthy individual is described by way of example.

10 cc of peripheral blood are taken from a 45 years old male patient; the blood is centrifuged so as to separate the plasma (containing the circulating free DNA) from the corpuscular component (lymphocytes and erythrocytes). In this patient, 14 uL of cfDNA are extracted at a concentration of 2.36 ng/uL, starting from 4 ml of plasma. 20 ng of cfDNA are used to make what is called “NGS library” that is a set of DNA fragments which are associated to a barcode (a synthetic DNA sequence) that defines the specimen in an unambiguous manner. On the basis of the read concentration (3390 pM), such a library is mixed (pooling) with the libraries obtained from other specimens (each of which will have a different barcode). The cfDNA is sequenced and the mutations in the panel of genes and hotspots of Table 1 (lung) are analysed; the mutation p.G12D of the KRAS gene at 0.49% is found.

After 6 months the same analysis is repeated and the same mutation p.G12D of the KRAS gene at 1.05% is found; the instability index is calculated with the formula

$${I}_{G} = {\sum }_{j = 1}^{n} \frac{Δ {F}_{j}}{n}$$

and the value 0,047 is obtained. Since the index value is below the 0.1 threshold it is recommended to repeat the test after 6 months.

TABLE 1

| | LUNG cfDNA HOTSPOTS |
|---|---|
| Gene | Amino-acid change |
| NRAS | p.Q61L p.Q61K, p.A59T, p.G13V, p.G13D, p.G13Y, p.G13V, p.G13A, |
| | p.G13N, p.G13R, p.G13C, p.G13S, p.G12E, p.G12D, p.G12P, |
| | p.G12Y, p.G12A, p.G12V, p.G12N, p.G12R, p.G12C, p.G12S |
| ALK | p.R1275L, p.R1275Q , p.F1245L, p.F1245L, p.F1245C, p.F1245I, |
| | p.F1245V, p.L1196Q, p.L1196M, p.V1180L, p.F1174L, p.F1174L, |
| | p.F1174C, p.F1174S, p.F1174I, p.F1174V, p.F1174L, p.I1171N, |
| | p.I1171N, p.I1171T, p.C1156Y, p.L1152P, p.L1152R, |
| | p.T1151_L1152insT, p.G1128A |
| PIK3CA | p.E542K, p.E545K, p.H1047R |
| ROS1 | p.L1951M |
| EGFR | p.E709K, p.E709A, p.G719C, p.G719S, p.G719A, |
| | p.K745_E746insIPVAIK, p.E746_A750delELREA, |
| | p.E746_A750delELREA, p.E746_T751A, p.E746_S752V, |
| | p.L747_E749delLRE, p.L747_A750P, p.L747_T751P, |
| | p.L747_S752delLREATS, p.L747_T751delLREAT, p.L747_P753S, |
| | p.S768I, p.V769_D770insASV, p.D770_N771insSVD, |
| | p.H773_V774insH, p.H773_V774insNPH, p.T790M, p.C797S, |
| | p.E709_T710 > D, p.E709_T710 > A, p.E709_T710 > G, p.E709H, |
| | p.E709G, p.E709V, p.G719D, p.H835L, p.P848L, p.L858R, p.L861Q |
| MET | p.T1010I, p.Y1021N, p.Y1021F, p.L982_D1028del, |
| | p.L982_D1028del, X1010_splice, p.H1112Y, p.H1112L, p.H1112R, |
| | p.Y1248H, p.Y1248C, p.Y1253N, p.Y1253H, p.Y1253D, p.M1268V, |
| | p.M1268T, p.M1268I |
| BRAF | p.V600E, p.G469V, p.G466V, p.Y472C, p.L597V, p.G469A, p.G469L |
| KRAS | p.Q61H, p.Q61R, p.Q61L, p.G13D, p.G13C, p.G12V, p.G12D, |
| | p.G12A, p.G12F, p.G12C, p.G12S, p.G12R |
| MAP2K1 | p.F53I, p.F53L, p.F53L, p.F53L, p.K57Q, p.Q56P, p.K57T, p.K57N, |
| | p.P124S, p.P124Q, p.P124L, p.E203K, p.E203V |
| TP53 | p.R337L, p.R283P, p.R282W, p.R280I, p.C277F, p.R273H, p.R273L, |
| | p.R273P, p.R273C, p.R249S, p.R249S, p.R249M, p.R248Q, |
| | p.R248L, p.R248W, p.G245V, p.G245C, p.C242F, p.M237I, |
| | p.Y234C, p.Y220C, p.H214R, p.Y205C, p.H179R, p.C176F, |
| | p.C176Y, p.R175H, p.V173L, p.Y163C, p.A159V, p.R158L, p.V157F, |
| | p.G154V, p.T125T |
| ERBB2 | p.A775_G776insYVMA |

### EXAMPLE 2

### Breast and Ovarian Tumors

The test for evaluation of the genetic stability and identification of the prodromal phase of the oncogenesis which carries out the method according to the invention is applied in a particular way to women who undergo, or have undergone in the past, hormone replacement therapies, contraception, or ovarian stimulation.

Moreover, it can be advantageously used in other specific cases of monitoring and prevention, for example prevention program for women who carry hereditary BRCA 112 mutation, with high risk to develop the uterine or ovarian tumor.

The panel of genes and mutations used in this case includes 10 genes and 159 hotspot, listed in the table (see Table 2).

An application of the method for evaluation of the genetic instability with regard to the panel related to breast-ovarian tumors for a healthy individual is described by way of example.

10 cc of peripheral blood are taken from a 57 years old female patient; the blood is centrifuged so as to separate the plasma (containing the circulating free DNA) from the corpuscular component (lymphocytes and erythrocytes). In this patient, 14 uL of cfDNA are extracted at a concentration of 1.71 ng/uL, starting from 4 ml of plasma. 20 ng of cfDNA are used to make what is called “NGS library” that is a set of DNA fragments which are associated to a barcode (a synthetic DNA sequence) that defines the specimen in an unambiguous manner. On the basis of the read concentration (3240 pM), such a library is mixed (pooling) with the libraries obtained from other specimens (each of which will have a different barcode). The cfDNA is sequenced and the mutations in the panel of genes and hotspots of Table 2 (breast and ovaries) are analysed; the mutation p.H1047R of the PIK3CA gene at 0.57% is found.

After 6 months the same analysis is repeated and the mutations p.H1047R of the PIK3CA gene at 1.75% are found and a new mutation p.R175H of the TP53 gene at 0.34% is found; the instability index is calculated with the formula

$${I}_{T} = {\sum }_{k = 1}^{n} \frac{Δ {F}_{k}}{n}$$

and the value 0.152 is obtained. Since the index value is over the 0.1 threshold, it is recommended to repeat the test after 3 months.

TABLE 2

| | BREAST cfDNA HOTSPOTS |
|---|---|
| Gene | Amino-acid change |
| SF3B1 | p.K700E |
| | p.N345K, p.C420R, p.E453K, p.E542K, p.E545Q, p.E545K, |
| | p.E545A, p.E545G, p.Q546K, p.Q546R, p.Q546P, p.E726K, |
| | p.M1043V, p.M1043I, p.H1047Y, p.H1047R, p.H1047L, |
| PIK3CA | p.G1049R |
| FBXW7 | p.D600Y, p.S582L |
| | p.E380Q, p.V392I, p.S463P, p.Y537N, p.Y537C, p.Y537S, |
| ESR1 | p.D538G |
| EGFR | p.H835L, p.P848L, p.L858R, p.L861Q |
| | p.G13D, p.G13C, p.G12A, p.G12D, p.G12F, p.G12V, |
| KRAS | p.G12R, p.G12C, p.G12S |
| AKT1 | p.E17K |
| | p.E286G, p.E286K, , p.E285K, p.R283P, p.R282G, p.R282W, |
| | p.R280I, p.R280K, p.R280T, p.G279E, p.P278R, p.P278L, |
| | p.P278A, p.P278S, p.P278T, p.C277F, p.C275Y, p.V274L, |
| | p.V274F, p.R273H, p.R273L, p.R273P, p.R273C, p.V272L, |
| | p.V272M, p.G266E, p.G266V, p.G266R, p.G262V, p.E258K, |
| | p.P250L, p.R249S, p.R249K, p.R249M, p.R248Q, p.R248L, |
| | p.R248W, p.M246V, p.G245D, p.G245V, p.G245C, p.G245S, |
| | p.G244D, p.G244V, p.G244C, p.G244S, p.C242F, p.C242Y, |
| | p.S241F, p.S240G, p.C238F, p.C238Y, p.M237I, p.Y234C, |
| | p.Y220C, p.Y220H, p.V216M, p.H214R, p.R213Q, p.R213L, |
| | p.V197M, p.I195T, p.L194R, p.H193R, p.H193Y, p.P191del, |
| | p.P190L, p.P177_C182del, p.H179R, p.H179L, p.H179Y, |
| | p.C176F, p.C176Y, p.R175H, p.R175L, p.R175C, p.V173L, |
| | p.V173M, p.V172F, p.R158H, p.R158L, p.V157F, p.R156P, |
| | p.G154V, p.P152L, p.P151H, p.P151S, p.P151T, p.L145P, |
| | p.C141Y, p.C141R, p.A138V, p.C135W, p.C135F, p.C135Y, |
| TP53 | p.K132R, p.K132E |
| ERBB2 | p.L755M, p.L755S |
| | p.R103G, p.V104M, p.V104M, p.V104M, p.V104L, p.V104L, |
| ERBB3 | p.V104L, p.G284R, p.G284R, p.G284R, p.G284R, p.D297Y, |
| | p.D297Y, p.T355I, p.T355I, p.E928G |

### Example 3

### Colon and Rectal Tumors

The evaluation of the genetic stability and identification of the prodromal phase of the oncogenesis related to the category of colon and rectal tumors includes the periodical analysis of 14 genes and 246 hotspots, as specified in Table 3, which lists all the genes currently involved with the respective hotspots.

The neoplasia that involves the colorectal system often develops as the evolution of a benign lesion, such as adenomatous polyposis, in the intestinal mucous membrane.

The formation of neoplasia can be fostered by some risk factors, like obesity or a diet rich in calories and fats and low in fiber, or genetic factors, for example, a family history of the pathology. Moreover, the age, chronic intestinal inflammatory pathologies and medical history of polyps can likewise contribute and increase the probability of the onset of the tumor.

The time it takes for a benign neoplasm to become malignant is very often long (7 to 15 years), and such evolution can be advantageously followed with the application of periodical tests and the consequent evaluation of the risk status provided by the method according to the invention.

An application of the method for evaluation of the genetic instability with regard to the panel related to colorectal tumors for a healthy individual is described by way of example.

10 cc of peripheral blood are taken from a 65 years old male patient; the blood is centrifuged so as to separate the plasma (containing the circulating free DNA) from the corpuscular component (lymphocytes and erythrocytes). In this patient, 14 uL of cfDNA are extracted at a concentration of 1.49 ng/uL, starting from 4 ml of plasma. 20 ng of cfDNA are used to make what is called “NGS library” that is a set of DNA fragments which are associated to a barcode (a synthetic DNA sequence) that defines the specimen in an unambiguous manner. On the basis of the read concentration (8130 pM), such a library is mixed (pooling) with the libraries obtained from other specimens (each of which will have a different barcode). The cfDNA is sequenced and the mutations in the panel of genes and hotspots of Table 1 (lung) are analysed; a mutation p.R1450Ter of the APC gene at 0.15% is found.

After 6 months the same analysis is repeated and the same mutation p.R1450* of the APC gene at 1.05% is found; the instability index is calculated with the formula

$${I}_{G} = {\sum }_{j = 1}^{n} \frac{Δ {F}_{j}}{n}$$

and the value 0.026 is obtained. Since the index value is below the 0.1 threshold, it is recommended to repeat the test after 6 months.

TABLE 3

| | COLON cfDNA HOTSPOTS |
|---|---|
| Gene | Amino-acid change |
| NRAS | p.Q61L, p.Q61R, p.Q61K, p.G13V, p.G13V, p.G13A, p.G13D, p.G13Y, |
| | p.G13N, p.G13S, p.G13R, p.G13C, p.G12E, p.G12V, p.G12D, p.G12A, |
| | p.G12P, p.G12Y, p.G12N, p.G12S, p.G12C, p.G12R |
| CTNNB1 | p.S33Y, p.G34V, p.T41A, p.T41I, p.S45P, p.S45F |
| PIK3CA | p.E542K, p.E545Q, p.E545K, p.E545A, p.E545G, p.Q546K, p.Q546R, |
| | p.Q546P, p.M1043V, p.M1043I, p.H1047Y, p.H1047R, p.H1047L, |
| | p.G1049R |
| FBXW7 | p.R689W, p.D600Y, p.S582L, p.W526R, p.R505C, p.R479Q, p.R465H, |
| | p.R465C |
| APC | p.R805Ter, p.R876Ter, p.Y935Ter, p.R1114Ter, p.S1234fs, p.Q1291Ter, |
| | p.Q1294Ter, p.Q1303Ter, p.E1306Ter, p.I1307fs, p.E1309fs, p.E1309fs, |
| | p.E1309Ter, p.E1309fs, p.E1309fs, p.G1312Ter, p.E1353Ter, p.P1361fs, |
| | p.Q1367Ter, p.P1372fs, p.P1373fs, p.Q1378Ter, p.E1379Ter, |
| | p.Q1406Ter, p.E1408Ter, p.S1411fs, p.R1450Ter, p.S1465fs, p.E1464fs, |
| | p.S1465fs, p.L1488fs, p.F1491fs, p.T1493fs, p.T1556fs, p.E1577Ter |
| EGFR | p.R451C, p.S464L, p.G465R, p.G465R, p.G465R, p.G465E, p.K467T, |
| | p.I491M, p.S492R, p.S492R |
| BRAF | p.V600E, p.L597V, p.D594G |
| KRAS | p.A146T, p.Q61H, p.Q61R, p.Q61L, p.G13D, p.G13C, p.G12A, p.G12D, |
| | p.G12V, p.G12F, p.G12R, p.G12C, p.G12S |
| AKT1 | p.E17K |
| MAP2K1 | p.F53I, p.F53L, p.F53C, p.F53L, p.Q56P, p.K57Q, p.K57T, p.K57N, |
| | p.E203K, p.E203V |
| TP53 | p.E286G, p.E286K, p.E285K, p.R283P, p.R282W, p.R282G, p.R280I, |
| | p.R280K, p.R280T, p.G279E, p.P278R, p.P278L, p.P278A, p.P278S, |
| | p.P278T, p.C277F, p.C275Y, p.V274L, p.V274F, p.R273H, p.R273L, |
| | p.R273P, p.R273C, p.V272L, p.V272M, p.G266E, p.G266V, p.G266R, |
| | p.G262V, p.E258K, p.P250L, p.R249S, p.R249K, p.R249M, p.R248Q, |
| | p.R248L, p.R248W, p.M246V, p.G245D, p.G245V, p.G245S, p.G245C, |
| | p.G244D, p.G244V, p.G244C, p.G244S, p.C242F, p.C242Y, p.S241F, |
| | p.S240G, p.C238F, p.C238Y, p.M237I, p.Y234C, p.Y220C, p.Y220H, |
| | p.V216M, p.H214R, p.R213Q, p.R213L, p.V197M, p.I195T, p.L194R, |
| | p.H193R, p.H193Y, p.P191del, p.P190L, p.P177_C182del, p.H179R, |
| | p.H179L, p.H179Y, p.C176F, p.C176Y, p.R175H, p.R175L, p.R175C, |
| | p.V173L, p.V173M, p.V172F, p.R158L, p.R158H, p.V157F, p.R156P, |
| | p.G154V, p.P152L, p.P151H, p.P151S, p.P151T, p.L145P, p.C141Y, |
| | p.C141R, p.A138V, p.C135W, p.C135F, p.C135Y, p.K132R, p.K132E |
| ERBB2 | p.S310F, p.S310Y, p.L755M, p.L755S, p.E770_A771insAYVM, p.G776V, |
| | p.V777L, p.V842I, p.R896C |
| SMAD4 | p.A118V, p.E330A, p.D351G, p.P356L, p.R361C, p.R361H, p.G386D, |
| | p.G510V |
| GNAS | p.R201C, p.R201S, p.R201H, p.R201L, p.Q227R |

It is understood that what above has been described as a pure not limiting example. Therefore, possible changes and variants of the invention are considered within the protective scope granted to the present method, as described above and claimed below.

### EXAMPLE 4

### General Study on the Basics of the Method cfDNA Extraction

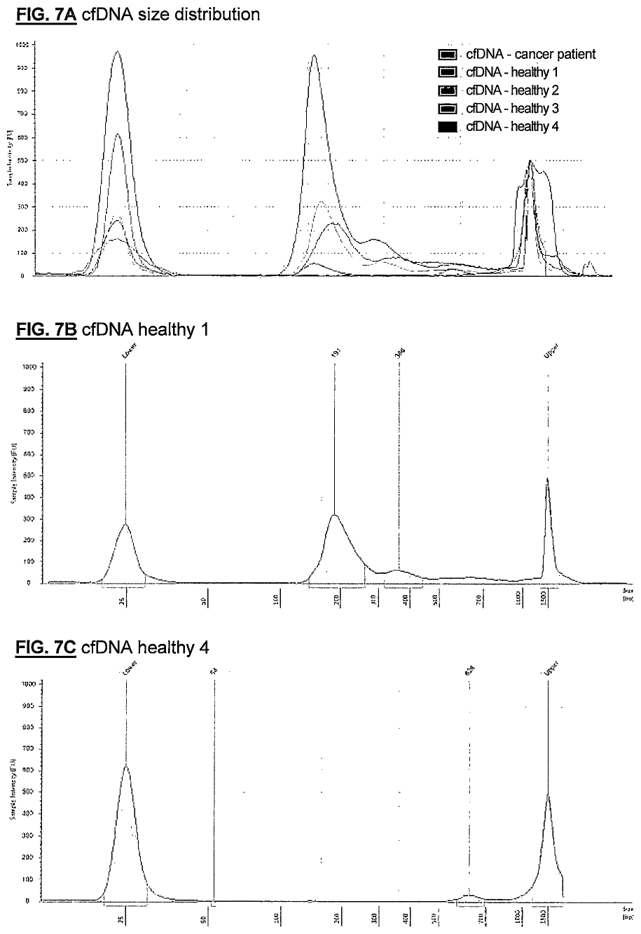

Blood samples were collected in either EDTA or into StreckTM DNA tubes. The plasma fraction was separated from the blood cells by two consecutive rounds of centrifugation for 30 min at room temperature at 1600xg. The collected plasma was aliquoted and stored at −80° C. until use. cfDNA was extracted from plasma volumes ranging from 0.4 to 5.5 ml using the MagMax Cell-Free Total Nucleic Acid Isolation Kit (Thermo Fisher Scientific) according to manufacturers' instructions. The cfDNA quantity was assessed with the dsDNA HS assay kit by the Qubit 2.0 Fluorometer (Thermo Fisher Scientifc). cfDNA quality was assessed with the Agilent High Sensitivity D1000 ScreenTape System (Agilent Technologies). Only cfDNA samples with a clear fragment size peak between 140-200 bp (FIGS. 7A-7C) were considered for analysis.

### NGS Library Preparation

NGS libraries were prepared from 2.5-105.5 ng of cfDNA following the HeliSmoker, HeliGyn and HeliSafe workflows (patented by The Bioscience Institute), based on the Oncomine™ Lung cfDNA Assay, the Oncomine™ Breast cfDNA Research Assay v2 and the Oncomine™ Pan-Cancer Cell-Free Assay (Thermo Fisher Scientific), respectively. Briefly, a two-cycle multiplex touch-down PCR reaction with a temperature range from 64° C. to 58° C. was performed in a total volume of 30 ul to amplify target regions and introduce unique molecular identifiers to the PCR products. The resulting tagged amplicons of around 100-140bp length were then cleaned up using AmpureXP beads (Agencourt) at a bead to sample ratio of 1.5× and purified products were eluted in 24 ul low TE buffer. A second round of PCR (18 cycles) was performed in a total volume of 50 ul to amplify the purified amplicons and introduce Ion Torrent™ Tag-Sequencing adapters containing sample-specific barcodes. The resulting library of target DNA fragments was purified by performing a two-step cleanup using AmpureXP beads (Agencourt) at a bead to sample ratio of 1.15× and 1.0×, respectively. The purified libraries were then diluted 1:1000 and quantified by qPCR using the Ion Universal Quantitation Kit (Thermo Fisher Scientific). The quantified stock libraries were then diluted to 100pM for downstream template preparation.

### Sequencing

NGS libraries were sequenced on an Ion S5™ instrument (Thermo Fisher Scientific) using semiconductor sequencing technology. Briefly, sequencing runs were planned on the Torrent Suite Software™ v5.8, libraries were pooled and loaded on an Ion 540™ chip using the Ion Chef™ instrument (Thermo Fisher Scientifc). The loaded chip was then inserted into the initialized Ion S5™ instrument and sequenced using 500 flows of the standard samba flow-order. Raw data was processed automatically on the Torrent Server™ and aligned to the reference hg19 genome. QC was performed manually for each sample based on the following metrics; number of reads per sample >2′500′000 (for Oncomine™ Lung cfDNA Assay libraries), >4′000′000 (for Oncomine™ Breast cfDNA Research Assay v2 libraries)>15′000′000 (for Oncomine™ Pan-Cancer Cell-Free Assay libraries), on-target reads >90%, read uniformity >90%, median molecular coverage >500x, median read coverage >15′000. The sequencing data of the QC passing samples was then uploaded in BAM format to the Ion ReporterTM Analysis Server for variant calling and annotation.

### Data Analysis

Variant calling was performed on Ion Reporter™ Analysis Software v5.6 using the Oncomine TagSeq Pan-Cancer Liquid Biopsy w2.0 and Oncomine TagSeq Breast v2 Liquid Biopsy w2.0 workflows. The alignment pipeline also included signalling processing, base calling, quality score assignment, adapter trimming, PCR duplicate removal and control of mapping quality. Local re-alignment, duplicate removal and quality base score recalibration were as well carried out using the Genome Analysis Toolkit (GATK) (McKenna et al., 2010). Coverage metrics for each amplicon was obtained by running the Coverage Analysis Plugin software v5.6.1 (Thermo Fisher Scientific). Identified variants were only considered if the variant had a molecular coverage of at least three, indicating that the variant was detected in three independent template molecules. This strategy allowed us to call specific somatic mutations down to 0.01% molecular allele fraction. Finally, all candidate mutations were manually reviewed using the Integrative Genomics Viewer (Thorvaldsdottir, Robinson, & Mesirov, 2013).

### Results

Plasma Volume and cfDNA amount define LOD for variant calling

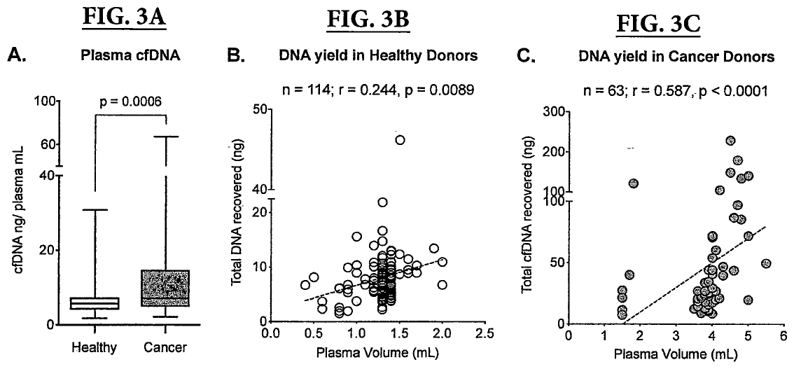

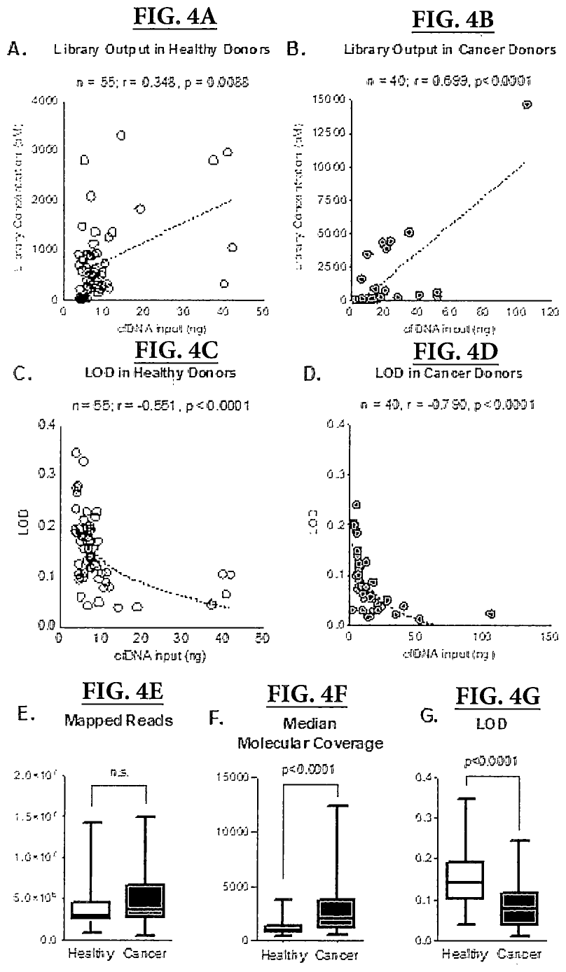

First, we attempted to establish a solid workflow for the extraction of cfDNA from plasma of either healthy individuals or cancer patients. Table 4 summarizes the analyzed cohort characteristics. After collection of peripheral whole blood using either standard EDTA or commercial vessels containing a preservative solution able to prevent nucleated cell lysis and therefore contamination of cfDNA with cellular DNA, we proceeded with DNA extraction using a magnetic beads-based kit as described in detail in the Materials and Methods section. The amount of plasma used varied between 0.4 and 2.0 ml in healthy individuals and 1.5 and 5.5 ml in cancer patients (FIGS. 3A-3C). Typically, more plasma was available for analysis in the cancer patient cohort because 2×10 ml of whole blood was collected for each patient. Conversely, only 1×10 ml whole blood was drawn from healthy individuals, part of which was used for other analyses. As previously observed (first in 1977 (Leon, Shapiro, Sklaroff, & Yaros, 1977), total cfDNA concentration in plasma was significantly higher in cancer patients compared to healthy subjects (p=0.0006, FIG. 3A). A significant correlation between plasma input and total cfDNA yield was observed in samples collected from healthy donors (r=0.244, p=0.0089, FIG. 3B) and cancer patients (r=0.587, p<0.0001, FIG. 3C). Next, we compared preanalytical variables encountered during processing of healthy and cancer donor samples. First, we aimed to correlate the amount of cfDNA input versus library output expressed as concentration. NGS library concentration was significantly affected by cfDNA input in both healthy and cancer samples (r=0.348, p=0.0088 and r=0.699, p<0.0001 respectively, FIGS. 4A-4B). Notably, as healthy individuals generally present with lower levels of cfDNA as compared to cancer patients (FIG. 3A), limited DNA input was used for library preparation, often below the minimal manufactures' recommended amount (i.e. 10 ng). Higher amount of cfDNA directly translated into higher molecular coverage due to the increased number of unique template DNA molecules given a constant amount of mapped reads (FIGS. 4E-4F). Conversely the limit of detection (LOD), which indicates the lowest variant allelic frequency that can be reliably detected by the used analytical method, was significantly lower in cancer patients (FIG. 4G). We show that LOD is clearly affected by the cfDNA abundance in both healthy individuals and cancer patients (FIGS. 4C-4D) with an inverse correlation between these two variables. Thus, our data show that the amount of cfDNA has a direct impact on sequencing performance and LOD.

### cfDNA Profiling of Cancer Patients and Concordance with Tissue

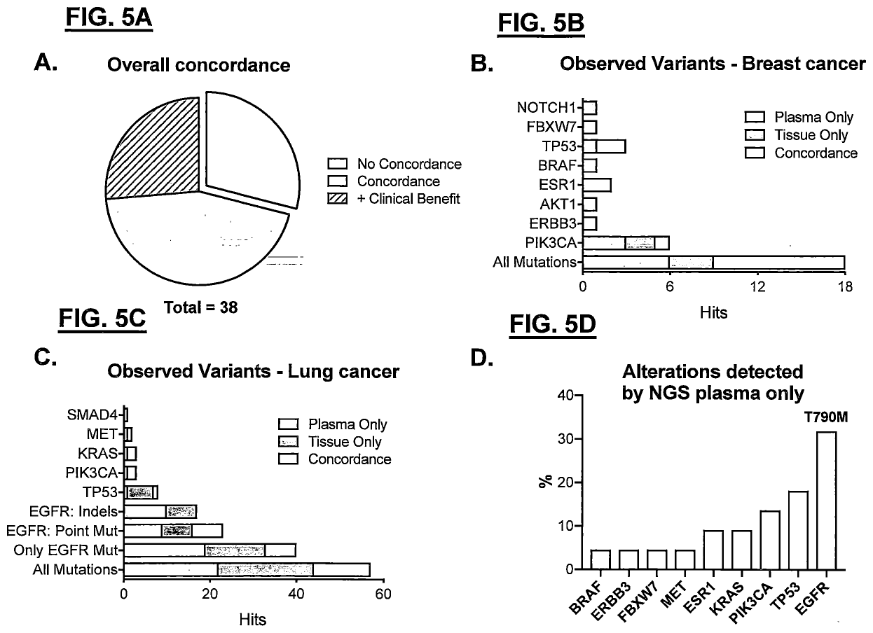

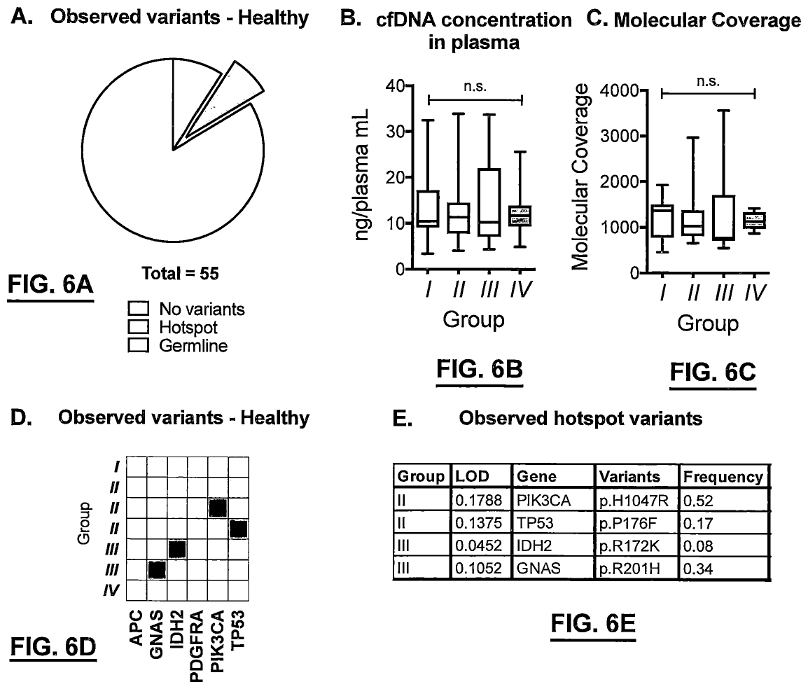

Previous studies proved the high analytic sensitivity of using unique molecular identifiers (UMI, (Kinde et al., 2011; Kivioja et al., 2011; Schmitt et al., 2012), when adopting synthetic DNA profiling. Here we attempted to investigate the concordance, in terms of corresponding detected variants, between circulating cfDNA and matched FFPE tissue from primary tumor or metastasis of the same patient. To this end, we analyzed cfDNA obtained from 8 breast cancer patients using the HeliGyn protocol (developed by the Bioscience Institute and based on the Oncomine cfDNA Breast v2 Assay) and 30 Non-Small Cell Lung Cancer (NSCLC) patients using the HeliSmoker protocol (developed by the Bioscience Institute and based on the Oncomine cfDNA Lung Assay) and sequentially compared it with the results obtained by sequencing FFPE tissue using a suitable Oncomine Assay (detailed about used gene panels in Supplementary Table 4). We used molecular barcoded ultra-deep sequencing (Tag Sequencing barcodes) to profile our liquid biopsy samples. As the gene content of the used panels for cfDNA and tissue was not entirely overlapping, we focused exclusively on clinically relevant mutations covered by both cfDNA and tissue panels (Supplementary Table 4). Our data highlight (FIG. 5A) a substantial level of concordance (71%) between the cfDNA and tissue generated mutation profiles. This suggests that cfDNA analyses reliably mimics tissue genomic features. For 26% of the samples showing a concordant result, additional clinically relevant mutations (FIG. 5D, e.g. T790M in EGFR) were detected through liquid biopsy (FIG. 5A, “plus Clinical Benefit”). The most frequently reported variants were mutations occurring within the coding region of PIK3CA (33% of all mutations considered) for breast cancer and EGFR (70% of all mutations considered) for NSCLC specimens (FIG. 5D). All mutations detected are summarized in a concordance matrix (Supplementary FIG. 5B-5C for breast and lung cancer samples, respectively). The time interval between tissue and blood collection ranged from 0 to 70 months, suggesting that tumor evolution and not only tumor heterogeneity could be the underlying reason for incongruence between tissue and liquid biopsy analysis (Supplementary FIG. 5C). Among mutations detected by plasma only, the T790M resistance mutation was the most frequent (32% of all mutations detected by plasma and not my tissue NGS analysis, FIG. 3D). These data confirm the potential clinical value of using liquid biopsy in parallel to tissue biopsy, particularly for detecting mutations that are relevant for acquired therapy resistance, as well as the effectiveness of our testing strategy.

### cfDNA Profiling of Heathy Individuals

Finally, we attempted to profile cfDNA of individuals that were healthy (as above defined) at the time of blood collection. Our patient cohort comprised n=106 women that underwent a control screening mammography test and had been followed up regularly up to 10 years later (Table 4). Blood was collected at the same time of the mammography screening. For this study we divided the healthy individuals into four groups based on clinical status at follow-up. Individuals belonging to group 1 (n=25) did not develop any breast cancer or other malignancies during follow-up time. In group 2 individuals (n=52) experienced fibrocystic breast changes such as fibroadenoma and hyperplasia during follow-up time. Donors allocated to group 3 (n=15) developed breast cancer, while in group 4 (n=14) they developed a solid tumor other than breast cancer. The results of the profiling are summarized in FIG. 4. As reported in the first paragraph, we successfully achieved cfDNA extraction from all plasma samples, with values ranging from 1.7 to 30.8 ng/ml of cfDNA (FIG. 1A). Based on recovery rate and quality of cfDNA (described in the Material and Methods section, Supplementary FIG. 1), we selected 55 samples for further analysis (group I=12/24; group II=23/52; group III=11/15; group IV=9/14; total=55/106), obtaining LOD ranging from 0.04 to 0.34 (FIG. 2C, Supplementary FIG. 1D). We sequenced the isolated cfDNA using the HeliGyn workflow (Supplementary Table 4) and we selected 6 samples for a broader mutational analysis using the HeliSafe protocol (based on the Oncomine cfDNA Pan-Cancer Assay, Supplementary Table 4, details in materials and methods). No genetic alterations were found in the cfDNA of most healthy individuals (84%) (FIG. 4A). Yet, in 7 of the 55 cases analyzed, we detected clinically relevant gene mutations, specifically six known germline variants observed at allelic frequencies above 40% and four known cancer hotspot mutations (FIG. 4D, E). Importantly, no significant difference has been observed between the four groups in terms of pre-analytical variables, including cfDNA concentration in plasma or achieved molecular coverage (FIG. 4B-C). In conclusion, our results provide evidence that genetic alterations can be detected in healthy individuals by analyzing cfDNA.

### DISCUSSION

Liquid biopsy has recently gained substantial attention in the field of cancer diagnostics. A growing body of evidence supports ctDNA-based analysis of cancer-associated hotspot mutations as a cost-effective and highly sensitive tool, complementary to tissue molecular profiling (Forshew et al., 2012; Kimura et al., 2006; Lanman et al., 2015; Narayan et al., 2012; Newman et al., 2016). Ambitious efforts are currently placed towards the implementation of liquid biopsy as an early cancer detection method (i.e. before cancer-related symptoms occur) and has already been applied to detected mutations in early stage tumors (Abbosh et al., 2017; Cohen et al., 2017; 2018; Phallen et al., 2017). Early diagnosis possibly equals to a better disease outcome, however large-scale validation studies are required to better understand the full potential and the limitations of this application of liquid biopsy (Cree et al., 2017). The screening of pre-cancerous lesions in asymptomatic individuals is hindered by several challenges. Namely, the number of mutant ctDNA molecules present in plasma is mostly proportional to tumor burden (Bettegowda et al., 2014), rendering detection particularly problematic in patients with localized cancer and healthy individuals. Another challenge is represented by the lack of knowledge regarding the molecular basis of tumor initiation. Several studies have reported the detection of somatic mutations and related clonal expansion in healthy tissue (Aghili, Foo, DeGregori, & De, 2014; Beane et al., 2017; Krimmel et al., 2016; Martincorena et al., 2015) associated with age and tissue proliferative rate (Yizhak et al., 2018). Some of these mutations were shown to increase the risk of developing cancer (Genovese et al., 2014; Jaiswal et al., 2014). The Pre-Cancer Genome Atlas will significantly improve our understanding of the role of pre-cancerous lesions in early stages of tumor formation, improving the specificity of early detection screening. Liquid biopsy can be used as minimally invasive detection method to characterize events of progression from normal tissue to cancerous development in longitudinal studies in healthy volunteers. At present liquid biopsy is mainly used in advanced cancer patients, however the Circulating Cell-free Genome Atlas (CCGA) study (GRAIL) and the development of early screening methods as CancerSEEK are opening the way for cfDNA testing in healthy individuals and early stage tumor patients. Our work aimed to contribute to this field by investigating the technical feasibility of using liquid biopsy for screening healthy individuals. Our cohort comprise 114 individuals clinically healthy at blood collection, as well as 63 patients with diagnosed breast or lung cancer. As expected, cfDNA concentration was significantly lower in healthy individuals compared to cancer patients (FIG. 1A), with cfDNA concentrations ranging from 1 to 16 ng m1⁻¹ in plasma for healthy individuals (with the exception of one sample which had a concentration of 30 ng m1⁻¹ in plasma), consistently with previously published results (Mouliere, Messaoudi, Pang, Dritschilo, & Thierry, 2014; Mouliere et al., 2011). To overcome the challenges associated with low input material as well as enabling the detection of low frequency mutations, we have implemented molecular barcoding and ultra-deep sequencing (Kinde et al., 2011; Kivioja et al., 2011; Newman et al., 2016; Schmitt et al., 2012, reviewed in Salk, Schmitt, & Loeb, 2018). We have confirmed the reliability and accuracy of our method by matched genomic analysis of tissue and plasma samples (concordance of 71% for our cohort of breast and lung cancer patients, FIG. 3A). As expected, likely due to tumor tissue heterogeneity further fostered by the time interval occurring between tissue and blood collection, we did not observe perfect concordance (Supplementary FIG. 3C). These results are in line with previous studies reporting sensitivity between 65% and 98% (Oxnard et al., 2016; Sacher et al., 2016; Schiavon et al., 2015; Thierry et al., 2014, reviewed in Wan et al., 2017). We could successfully isolate cfDNA and produce functional NGS libraries from as little as 0.9 mL of plasma, however we observed higher LOD (FIG. 2G) in healthy donors compared to cancer patients due to higher availability of cfDNA in cancer patients. As healthy individuals present with lower levels of cfDNA compared to cancer patients (FIG. 1A), we recommend using higher volumes of plasma for cfDNA analysis from healthy donors. Importantly, this would allow for the detection of variants with low allelic frequency, which could be particularly relevant for discovering the presence of early genomic instability events (as shown by the four cancer hotspot mutations we identified, FIG. 4D). Through our analysis we detected genetic alterations in 7 out of 55 subjects with evaluable cfDNA that were considered clinically healthy at the time of liquid biopsy. Amongst these mutations, we found six germline variants and four cancer hotspot mutations. The observation of germline variants is a byproduct of our cIDNA analysis. Interestingly, many germline variants detected in our study are mutations in the coding region of TP53. Those patients might be recommended to have genetic counseling and upon decision of a trained certified geneticist to access early prevention programs. The four cancer hotspot mutations detected are recurrent genetic alterations, clinically classified as pathogenic or likely pathogenic. We detected cancer hotspot variants in individuals that were diagnosed with a benign breast condition (group II) or breast cancer (group III) up to 9 years later and at allelic frequencies ranging from 0.08 to 0.52% (FIG. 4D). Observing these mutations in the cIDNA of healthy donors might be considered as indirect evidence of genomic instability, as was shown for the PIK3CA p.H1047R variant (Miller, 2012). However, it was also observed that pathogenic TP53 mutations can be detected in the cfDNA of healthy controls (Fernandez-Cuesta et al., 2016) with no correlation to tumor insurgence. Therefore, the interpretation of these findings warrant caution and needs to be carefully considered before drawing any conclusion. Additional extensive prospective studies with long follow-up time and available tissue specimens for individuals that developed cancer will be required to address the specificity and sensitivity of liquid biopsy as a tool for early cancer detection. In conclusion, with this work we have established a rapid and reliable workflow that allowed us to interrogate cfDNA from healthy individuals to study genomic alterations with a limit of detection as low as 0.04% of allelic frequency. The interrogation cfDNA from the blood of healthy individuals could prove to be a prospective tool to detect signs of genomic instability and to better understand early events in tumor formation.

TABLE 4

| | |
|---|---|
| Patient characteristics (n = 177) | No. (%) |

Age (yrs)

| | | |
|---|---|---|
| Mean (SD) | 63 | (11) |

Sex (n)

| | | |
|---|---|---|
| Male | 19 | (11) |
| Female | 157 | (89) |

Clinical status at follow up (n)

| | | |
|---|---|---|
| No tumor (healthy) | 25 | (14) |
| Bening breast condition | 52 | (29) |
| Breast cancer | 24 | (14) |
| Lung cancer | 54 | (31) |
| Other tumors | 14 | (8) |
| Missing information (no follow up) | 8 | (5) |

Clinical status at blood collection (n)

| | | |
|---|---|---|
| No tumor (healthy) | 114 | (64) |
| Breast cancer | 9 | (5) |
| Lung cancer | 54 | (31) |

Follow up time from blood collection (yrs)

| | | |
|---|---|---|
| Healthy at blood collection - mean (range) | 8.3 | (1.1-10.5) |

Molecular analysis (n)

| | | |
|---|---|---|
| Plasma - cfDNA extraction | 177 | (100) |
| Plasma - NGS analysis | 93 | (53) |
| Tissue - NGS analysis | 38 | (21) |

FIGS. 3A, 3B, and 3C show total cfDNA yield of plasma samples deriving from healthy donors or cancer patients: FIG. 3A shows cfDNA concentration in the plasma of healthy individuals compared to cancer patients (Mann-Whitney p=0.0006,). Median, interquartile range and minimum/maximum are shown in the boxplot. FIG. 3B shows correlation of plasma volume and the total cfDNA output in healthy donors (n=114, Spearman r=0.244, p=0.0089). FIG. 3C shows orrelation between the plasma volume and the total cfDNA output in cancer patients (n=63, Spearman r=0.587, p<0.0001).

Comparison of preanalytical variables from healthy and cancer donor samples is shown in FIGS. 4A-4G. Correlation of library concentration and input of cfDNA in healthy individuals (n=55, Spearman r=0.348, p=0.0088) and cancer patients (n=40, Spearman r=0.699, p<0.0001) is shown in FIGS. 4° -4B. Correlation of LOD and cfDNA input in healthy (n=55; Spearman correlation: r=−0.551, p<0.0001) and cancer donors (n=40; Spearman correlation: r=−0.790, p<0.0001) is shown in FIGS. 4C-4D. Mapped reads (F) of samples deriving from healthy and cancer donors (Mann-Whitney p=0.1422) are shown in FIG. 4E. Median molecular coverage (Mann-Whitney p<0.0001) and LOD (Mann-Whitney p<0.0001) in healthy and cancer donors is shown in FIGS. 4F-4G. Median, interquartile range and minimum/maximum are shown in the boxplot.

Concordance analysis of liquid and tissue biopsy in cancer patients is shown in FIGS. 5A-5D. Representation of the percentage of overall concordance of matched tissue and liquid biopsy is shown in FIG. 5A. “+Clinical benefit” refers to additional clinically relevant mutations that were detected through NGS analysis of liquid biopsy and not tissue biopsy (see “plasma only” in the next sections). No concordance was observed in 29% of the samples, whereas out of 71% concordant samples 26% carried additional clinically relevant mutations detected by plasma only (+Clinical Benefit). Number of observed variants for breast (B) and lung (C) cancer samples are shown in FIG. 5B and FIG. 5C. Only clinically relevant variants covered by both tissue and plasma NGS panels were considered for the analysis. Distribution of alterations detected by NGS analysis of plasma and not detected in tissue is shown in FIG. 5D. Amongst the clinically relevant mutations that were detected through NGS analysis of liquid biopsy and not tissue biopsy, the most frequent (32%) is T790M in EGFR.

Genetic alterations detected in the cfDNA of healthy individuals is shown in FIGS. 6A-6E. No genetic alteration was detected in 84% of the assayed samples, however we detected 6 germline and 4 hotspot variants in 7 different samples as shown in FIG. 6A. Preanalytical variables as cfDNA concentration in plasma (B) and median molecular coverage (C) in the four groups of healthy donors (Kruskal-Wallis p==0.9223 and p=0.7721, respectively) are shown in FIGS. 6B and 6C. Group I: healthy at follow-up time; group II: benign breast condition at follow-up time; group Ill: breast cancer at follow up time; group IV: a solid tumor other than breast cancer at follow up time. Median, interquartile range and minimum/maximum are shown in the boxplot. Mutational matrix indicating the variants detected in healthy individuals belonging to the four groups is shown in FIG. 6D. Each line represents a patient. Yellow squares represent hotspot variants, grey squares represent germline variants. FIG. 6E presents a table summarizing the hotspot variants detected in healthy individuals

cfDNA size distribution in healthy individuals and cancer patients is shown in FIGS. 7A-7C. cfDNA size distribution of a cancer patient (purple line) compared to healthy donors (yellow, red, blue, green line) is shown in FIG. 7A. cfDNA profile of a healthy donor (cfDNA healthy 1, red line in A) with clear fragment size peaks at 191 and 366bp is shown in FIG. 7B. cfDNA profile of a healthy donor (cfDNA healthy 4, green line in A) with no fragment size peaks at the expected range for cfDNA is shown in FIG. 7C. Only cfDNA samples with a clear fragment size peak between 140 — 200 bp were selected for NGS analysis.

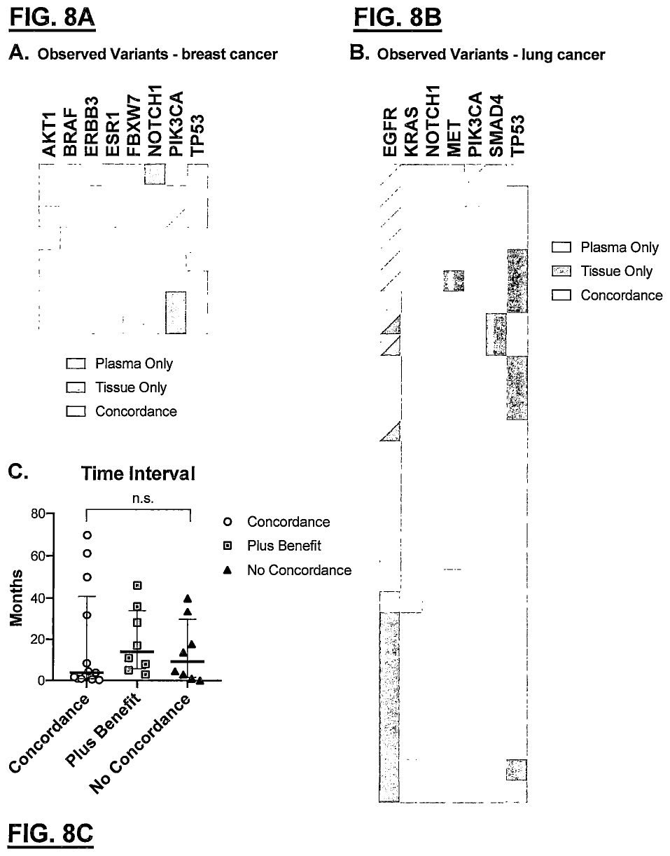

Concordance analysis of matched plasma and tissue samples from breast and lung cancer patients is shown in FIGS. 8A-8C. Concordance matrix of breast (A) and lung (B) cancer samples, each line represents a patient is shown in FIGS. 8A and 8B. Blue squares represent variants detected by plasma only, green squares represent variants detected by tissue only, yellow squares represent variants detected by both plasma and tissue. FIG. 8C presents time interval between blood and tissue collection (n =29, Kruskal-Wallis p=0.4325; values missing for 9 out of 38 samples, median for concordance group=3.8 months, median for plus benefit group=12 months, median for no concordance group=9 months). Median and minimum/maximum are represented in the plot.

SUPPLEMENTARY TABLE 1
Supplementary Table 1: Gene content of the used Oncomine
NGS panels for cfDNA and tissue analysis.

| Breast | Lung | Pancancer | Solid Tumour | Focus DNA |
|---|---|---|---|---|
| cfDNA v2 | cfDNA | cfDNA | DNA Kit | Assay |
| AKT1 | ALK | AKT1 | AKT1 | AKT1 |
| EGFR | BRAF | ALK | ALK | ALK |
| ERBB2 | EGFR | APC | BRAF | APC |
| ERBB3 | ERBB2 | AR | CTNNB1 | AR |
| ESR1 | KRAS | ARAF | DDR2 | BIRC2 |
| FBXW7 | MAP2K1 | BRAF | EGFR | BRAF |
| KRAS | MET | CCND1 | ERBB2 | BRCA1 |
| PIK3CA | NRAS | CCND2 | ERBB4 | CCND1 |
| SF3B1 | PIK3CA | CCND3 | FBXW7 | CDK4 |
| TP53 | ROS1 | CDK4 | FGFR1 | CDK6 |
| | TP53 | CDK6 | FGFR2 | CTNNB1 |
| | | CHEK2 | FGFR3 | DCUN1D1 |
| | | CTNNB1 | KRAS | DDR2 |
| | | DDR2 | MAP2K1 | EGFR |
| | | EGFR | MET | ERBB2 |
| | | ERBB2 | NOTCH1 | ERBB3 |
| | | ERBB3 | NRAS | ERBB4 |
| | | ESR1 | PIK3CA | ESR1 |
| | | FBXW7 | PTEN | FGFR1 |
| | | FGFR1 | SMAD4 | FGFR2 |
| | | FGFR2 | STK11 | FGFR3 |
| | | FGFR3 | TP53 | FGFR4 |
| | | FGFR4 | | GNA11 |
| | | FLT3 | | GNAQ |
| | | GNA11 | | HRAS |
| | | GNAQ | | IDH1 |
| | | GNAS | | IDH2 |
| | | HRAS | | JAK1 |
| | | IDH1 | | JAK2 |
| | | IDH2 | | JAK3 |
| | | KIT | | KIT |
| | | KRAS | | KRAS |
| | | MAP2K1 | | MAP2K1 |
| | | MAP2K2 | | MAP2K2 |
| | | MET | | MED12 |
| | | MTOR | | MET |
| | | MYC | | MTOR |
| | | NRAS | | MYC |
| | | NTRK1 | | MYCN |
| | | NTRK3 | | NF1 |
| | | PDGFRA | | NRAS |
| | | PIK3CA | | PDGFRA |
| | | PTEN | | PIK3CA |
| | | RAF1 | | RAF1 |
| | | RET | | RET |
| | | ROS1 | | ROS1 |
| | | SF3B1 | | SMO |
| | | SMAD4 | | |
| | | SMO | | |
| | | TP53 | | |

### REFERENCES

Cancer Genome Landscapes Science. 2013 Mar. 29; 339(6127): 1546-1558. Bert Vogelstein, Nickolas Papadopoulos, Victor E. Velculescu, Shibin Zhou, Luis A. Diaz, Jr., and Kenneth W. Kinzler*

Hereditary Cancer Risk Assessment: New Perspectives and Challenges for the Next-Gen Sequencing Era. Front Oncol. 2016; 6: 133. Israel Gomy

Principles in genetic risk assessment Ther Clin Risk Manag. 2005 Mar.; 1(1): 15-20. Pedro Viana Baptista

Predictive genomics: A cancer hallmark network framework for predicting tumor clinical phenotypes using genome sequencing data. Wang E. et al.

Mandel P, Metals P. Les acides nucleiques du plasma sanguin chez I′ homme [The nucleic acids in blood plasma in humans]. C R Seances Soc Biol Fil. 1948;142(3-4):241-243.

Stroun M, Anker P, Maurice P, Lyautey J, Lederrey C, Beljanski M. Neoplastic characteristics of the DNA found in the plasma of cancer patients. Oncology. 1989;46(5):318-322

Sausen M, Phallen J, Adleff V, Jones S, Leary R J, Barrett M T, Anagnostou V, Parpart-Li S, Murphy D, Kay Li Q, Hruban C A, Scharpf R, White J R, O′Dwyer P J, Allen P J, Eshleman J R, Thompson C B, Klimstra D S, Linehan D C, Maitra A, Hruban R H, Diaz L A Jr, Von Hoff D D, Johansen J S, Drebin J A, Velculescu V E. Clinical implications of qenomic alterations in the tumour and circulation of pancreatic cancer patients. Nat Commun. 2015 Jul. 7; 6:7686. doi: 10.1038/ncomms8686.

Hao T B, Shi W, Shen X J, et al. Circulating cell-free DNA in serum as a biomarker for diagnosis and prognostic prediction of colorectal cancer. Br J Cancer. 2014; 111(8):1482-1489.

Zonta E, Nizard P, Taly V. Assessment of DNA integrity, applications for cancer research. Adv Clin Chem. 2015;70:197-246

Chen, C. W. and Thomas, C. A. Jr. (1980) Recovery of DNA segments from agarose gels. Anal. Biochem.101, 339-41.

Marko, M. A. et al. (1982) A procedure for the large-scale isolation of highly purified plasmid DNA using alkaline extraction and binding to glass powder. Anal. Biochem. 121,382-7.

Boom, R. et al. (1990) Rapid and simple method for purification of nucleic acids. J. Clin. Microbiol. 28, 495-503.

Melzak, K. A. et al. (1996) Driving forces for DNA adsorption to silica in perchlorate solutions. J. Colloid Interface Sci. (USA) 181, 635-44.

Jones S, Anagnostou V, Lytle K, Parpart-Li S, Nesselbush M, Riley DR, Shukla M, Chesnick B, Kadan M, Papp E et al: Personalized genomic analyses for cancer mutation discovery and interpretation. Science translational medicine 2015, 7(283):283ra253.

Ng CK, Piscuoglio S, Geyer F C, Burke K A, Pareja F, Eberle C, Lim R, Natrajan R, Riaz N, Mariani O et al: The Landscape of Somatic Genetic Alterations in Metaplastic Breast Carcinomas. Clinical cancer research : an official journal of the American Association for Cancer Research 2017.

Gagan J, Van Allen E M: Next-generation sequencing to guide cancer therapy. Genome medicine 2015, 7(1):80.

Genomes Project C, Auton A, Brooks L D, Durbin R M, Garrison E P, Kang H M, Korbel J O, Marchini J L, McCarthy S, McVean G A et al: A global reference for human genetic variation. Nature 2015, 526(7571):68-74.

Ashworth A, Lord C J, Reis-Filho J S: Genetic interactions in cancer progression and treatment. Cell 2011, 145(1):30-38.

Richards S, Aziz N, Bale S, Bick D, Das S, Gastier-Foster J, Grody W W, Hegde M, Lyon E, Spector E et al: Standards and guidelines for the interpretation of sequence variants: a joint consensus recommendation of the American College of Medical Genetics and Genomics and the Association for Molecular Pathology. Genetics in medicine: official journal of the American College of Medical Genetics 2015, 17(5):405-424.

7.De Mattos-Arruda L, Caldas C: Cell-free circulating tumour DNA as a liquid biopsy in breast cancer. Molecular oncology 2016, 10(3):464-474.

8.Diaz LA, Jr., Bardelli A: Liquid biopsies: genotyping circulating tumor DNA. Journal of clinical oncology: official journal of the American Society of Clinical Oncology 2014, 32(6):579-586.

9.Pantel K, Diaz L A, Jr., Polyak K: Tracking tumor resistance using ‘liquid biopsies’. Nature medicine 2013, 19(6):676-677.

10.Garraway L A, Verweij J, Ballman K V: Precision oncology: an overview. Journal of clinical oncology : official journal of the American Society of Clinical Oncology 2013, 31(15):1803-1805.

11.Yohe S L, Carter A B, Pfeifer J D, Crawford J M, Cushman-Vokoun A, Caughron S, Leonard D G: Standards for Clinical Grade Genomic Databases. Archives of pathology & laboratory medicine 2015, 139(11):1400-1412.

12.Aaboud M, Aad G, Abbott B, Abdallah J, Abdinov 0, Abeloos B, Aben R, AbouZeid OS, Abraham NL, Abramowicz H et al: Search for triboson [Formula: see text] production in pp collisions at [Formula: see text] [Formula: see text] with the ATLAS detector. The European physical journal C, Particles and fields 2017, 77(3):141.

13.Dacheva D, Dodova R, Popov I, Goranova T, Mitkova A, Mitev V, Kaneva R: Validation of an NGS Approach for Diagnostic BRCA1/BRCA2 Mutation Testing. Molecular diagnosis & therapy 2015, 19(2):119-130.

Bettegowda C. Sausen M, Leary R J, Kinde I, Wang Y, Agrawal N, Bartlett B R, Wang H, Luber B, Alani R M et al: Detection of circulating tumor DNA in early- and late-stage human malignancies. Science translational medicine 2014, 6(224):224ra224.

1. A system for searching and identifying a genetic condition prodromal of the onset of solid tumors in a healthy subject including an evaluation cycle for the evaluation of of a genetic stability or instability condition and at least one repetition cycle, said repetition of such evaluation cycle, the repetition cycles periodically performed on the subject, with each cycle comprising:
taking a sample of biological material of the subject, isolating DNA from the biological material, amplifying and sequencing the isolated DNA;
verifying the presence of mutations selected in a predetermined set of genes of the sample under consideration, said set of genes and said mutations associated with the onset of solid tumors, the predetermined set of genes and selected mutations defined in view of anamnesis of the subject;
the predetermined set of genes including either a subset of the set of genes or hotspots connected to one or more solid tumors, or the entire set of genes connected to solid tumors;
verifying the frequency of mutations detected for each gene and for each evaluation cycle, the mutations chosen from the aforementioned selected mutations;
recording the mutations detected for each gene or group of genes and their frequency;
defining or updating a genetic instability index of the subject, either overall (I_{T}), or for a single gene (I_{G}), for each repetition cycle, based on the frequency of mutations detected and on the basis of an increase in the frequency of mutations, the genetic instability index (I_{T}, I_{G}) also defined on the basis of the increase in the frequency of mutations with respect to one or more previous evaluation cycles; and
evaluating, in each repetition cycle, the subject's entry into a genetic condition prodromal of the onset of one or more solid tumors or groups of solid tumors on the basis of a threshold value (I_{TS}, I_{GS}) of said genetic instability index (I_{T}, I_{G}), defined for each single gene or group of genes, based on the genetic instability index of the subject exceeding the threshold value; wherein
said system comprises a set of instructions of a computer program aimed at carrying out the evaluation cycle of a genetic stability or instability condition and at least one repetition cycle of said evaluation of a genetic stability or instability condition.

2. The system of claim 1, wherein said overall genetic instability index (IT) is defined as the summation of the relationships between a value (ΔFₖ) responsive to the increase in the observed mutations for each gene and the number of genes evaluated in consideration of the whole group of monitored genes.

3. The system of claim 1, wherein said index of genetic instability for single gene (IG) is defined as the summation of the relationships between a value (ΔFₖ) responsive to the increase in the observed mutations for each hotspot and the number of hotspots evaluated, in consideration of the whole group of monitored hotspots.

4. The system of claim 1, wherein the biological sample consists of a liquid biopsy, and the phase of verification of the presence of mutations in the predetermined set of genes is performed on a DNA sample isolated from said liquid biopsy and subsequently amplified and sequenced.

5. The system of claim 4, wherein the liquid biopsy is peripheral blood.

6. The system of claim 4, wherein the liquid biopsy is urine or spinal fluid.

7. The system of claim 4, wherein a fraction of cfDNA is sought in the DNA sample being analyzed and wherein the presence of mutations is verified in said cfDNA fraction.

8. The system of claim 7, wherein the ctDNA isolated from the liquid biopsy is also sought in the DNA sample being analyzed.

9. The system of claim 8, wherein, following the identification of circulating ctDNA, addressing information is sent to an early detection system.

10. The system of claim 8, wherein, following the identification of ctDNA in said DNA sample being analyzed, the presence of circulating tumor cells is sought in said liquid biopsy.

11. The system of claim 1, wherein the predetermined set of genes and selected mutations are defined in view of their connection to particular types of tumor.

12. The system of claim 1, wherein the repetition period is recalculated after each repetition cycle according to a value of said instability index (I_{T}, I_{G}) calculated in the current cycle and a value of the same as calculated in one or more previous cycles.

13. The system of claim 1, wherein a greater analysis sensitivity related to the monitored gene or set of genes is set in each repetition cycle, following an increase in the calculated value for said instability index (I_{T}, I_{G}) with respect to the value of the same index (I_{T}, I_{G}) calculated in one or more previous cycles.

14. The system of claim 1, wherein, in each of said evaluation and repetition cycles, germ DNA is also isolated and sequenced, and only the mutations present in cfDNA and not present in said germ DNA are considered for the subsequent calculation of the instability index.

15. The system of claim 14, wherein said germ DNA is sequenced with a same reading degree of the sequencing of the cited cfDNA.

16. The system of claim 14, wherein said germ DNA derives from the same liquid biopsy from which the cited cfDNA has been taken.
